SEQUENCE LISTING (4 sequences)

SEQ ID NO: 1 - DNA, 1820 bp, Homo Sapiens
ggcgctgggcggctgcggcgcgcggtgcgcggtgcgtagtctggagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggtacaatgca

SEQ ID NO: 2 - protein, 250 aa, Homo Sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWG

SEQ ID NO: 3 - DNA, 1810 bp, Homo Sapiens
ggcgctgggcggctgcggcgcgcggtgcgcggtgcgtagtctggagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggtacaatgcaaaaaaaaaaaaaaaaa

SEQ ID NO: 4 - protein, 376 aa, Homo Sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

(19) United States
(12) Patent Application Publication
(10) Pub. No.: US 2012/0202751 A1
(43) Pub. Date: Aug. 9, 2012

(54) METHODS OF MONITORING ENDOPLASMIC RETICULUM (ER) STRESS RESPONSE

(75) Inventors: Albert C. Koong, Los Altos, CA (US); Michael T. Spiotto, Chicago, IL (US); George P. Yang, Stanford, CA (US)

(73) Assignee: The Board of trustees of the Leland Stanford Junior University, Palo Alto, CA (US)

(21) Appl. No.: 13/389,765

(22) PCT Filed: Aug. 10, 2010
(86) PCT No.: PCT/US10/45080

§ 371 Date: Apr. 24, 2012

Related U.S. Application Data
(60) Provisional application No. 61/232,661, filed on Aug. 10, 2009.

(51) Int. Cl. A61K 38/07; A61K 49/00; A01K 67/027; C12N 5/10; C12Q 1/66

(52) U.S. Cl. 514/20.1; 435/349; 435/325; 435/350; 435/351; 435/363; 435/352; 435/353; 435/354; 435/355; 435/356; 435/357; 435/8; 800/9; 800/10; 800/12; 800/3

(57) ABSTRACT

Transgenic mammals, cells derived from the animals, and methods of using these to monitor the endoplasmic reticulum (ER) stress response are provided. In some embodiments, the methods allow for monitoring the ER stress response in real time. Some of the methods allow non-invasive in vivo visualization of ER stress response. Also provided are methods of screening molecules and/or treatment conditions for the ability to modulate the ER stress response, methods of treating diseases characterized by ER stress response activity, and methods of detecting the toxicity or therapeutic ratio of molecules that modulate the ER stress response.

This application claims the benefit of priority of U.S. Provisional Patent Application No. 61/232,661, filed Aug. 10, 2009, which is incorporated by reference in its entirety.

This work was supported in part by US National Cancer Institute grant PO1 CA67166. The government may have certain rights to the invention.

Cancer cells exist in harsh microenvironments that are governed by various factors including stromal cells, hypoxia, and nutrient deprivation. These microenvironmental stressors activate signaling pathways that affect a cancer cell's survival and response to therapy. These microenvironmental pressures interact during tumorigenesis to promote or inhibit tumor growth. For example, stromal cells may receive or transmit signals from or to cancer cells to promote angiogenesis, invasion and metastasis. Hypoxia activates pro-growth signaling pathways and destabilizes a cancer cell's genome to enhance tumorigenesis. By contrast, nutrients may become limited in the tumor microenvironment potentially inhibiting cell growth and/or activating cell death pathways. It remains difficult, however, to detect the real time activation of these downstream signaling pathways in primary tumors. Furthermore, the tumor microenvironment remains a poorly characterized stress on cancer cells due to its multifactorial nature.

By understanding how the tumor microenvironment may impact signaling pathways among similar primary tumors, one can gain insights into cancer biology and individualizing cancer therapy. One putative marker for the tumor microenvironment is ER (endoplasmic reticulum) stress which can be triggered within cancer cells by severe hypoxia or nutrient deprivation. These stressors lead to the abnormal accumulation of proteins within the ER. Cells respond to this ER stress by activating the unfolded protein response (UPR) that regulates transcription and translation of genes in order to maintain ER homeostasis. The UPR encompasses a three-pronged signaling network which includes the Inositol-requiring 1α (Ire1α)-X-box binding protein 1 (XBP1) pathway. The UPR interacts to promote cell survival or, if the ER stress is too great or too long, to activate cell death. The UPR is activated in tumors including high grade breast, colorectal, and pancreatic carcinomas. In addition, UPR proteins can promote tumor growth in both transplantable and primary tumor models. Thus, the UPR may link the tumor microenvironment to cancer cell death and survival as well as serve as a marker for the microenvironmental stressors occurring in primary tumors.

Accordingly, methods that identify conditions (e.g., molecules) that inhibit the ER stress response, and therefore the ability of cancer cells to adapt to and survive in harsh microenvironments, will be useful, for example, to develop efficacious therapeutic interventions. Similarly, methods that identify conditions that activate the ER stress response, further stress the cells, and overwhelm this stress-adaptive system will also be useful, for example, in developing new therapeutic interventions. Therefore, a need exists for both in vitro and in vivo methods of detecting modulation of the ER stress response and methods of screening for modulators of the ER stress response, among other things. In some embodiments, these methods allow real-time monitoring of the ER stress response. In some embodiments, the monitoring is non-invasive.

Provided are methods of detecting modulation of ER stress response and methods of screening for modulators of the ER stress response. The methods are based, at least in part, on the discovery that a XBP1 luminogenic reporter can be detected in real time, and non-invasively, in an in vivo model system of cancer. Activation of the ER stress response was observed within particular areas of the (primary) tumor microenvironment. Tumors were seen to display heterogeneous activation of the ER stress response, which was not based on genetic heterogeneity, but instead reflected unique tumor microenvironments.

A method of detecting modulation of ER stress response is provided. In some embodiments, the method comprises incubating a transgenic eukaryotic cell containing a heterologous luminogenic ER stress response reporter construct capable of directing expression of a luminogenic reporter under conditions suspected of modulating the ER stress response and detecting modulation of the expression of the luminogenic reporter, thereby detecting modulation of the ER stress response in the cell. In some embodiments, the conditions suspected of modulating the ER stress response comprise one or more physiological stimuli, such as low O₂, low glucose, or low pH. In some embodiments, the condition suspected of modulating the ER stress response includes incubating the cell with a test compound, such as a biologic or a small molecule. In some embodiments, the test molecule is a chemotherapeutic agent. In some embodiments, the cell is cultured in the presence of a second molecule, e.g., a molecule known to modulate the ER stress response, such as a proteasome inhibitor (e.g., bortezomib), brefeldin A, beta-mercaptoethanol, tunicamycin, 2-deoxy-d-glucose, dichloroacetic acid, thapsigargin, dithiothreitol, or combinations thereof. In some embodiments, the conditions suspected of modulating ER stress response include both one or more physiological stimuli and one or more test molecules. In some embodiments, increases or decreases in ER stress response, relative to control cells, are detected in response to a treatment.

In some embodiments, the transgenic eukaryotic cell used in the methods is a mammalian cell, such as a primate or rodent cell. In some embodiments, the cell is a rat or mouse cell.

In some embodiments, the transgenic eukaryotic cell is isolated from a transgenic mammal that comprises the luminogenic ER stress response reporter integrated in the genome of its germ cells. The cell may be cultured in vivo or ex vivo. In embodiments where the cell is cultured in vivo, the cell may be cultured autogenically, allogenically, or syngenically, for example. In some embodiments where the cell is cultured in vivo, the method allows the luminogenic reporter molecule to be detected non-invasively.

In some embodiments, the transgenic eukaryotic cell further comprises a mutation associated with an increased risk of a tumorigenic disease. The mutation may be a spontaneous de novo mutation or an engineered mutation. In some embodiments, the mutation is a gain of function mutation, such as an activated oncogene or a heterologous tumor transgene. In some embodiments, the mutation is a loss of function mutation, such as the loss of a tumor suppressor gene. In some embodiments, the transgenic eukaryotic cell expresses reduced levels of anti-apoptotic proteins. In some embodiments, the cell expresses increased levels of pro-apoptotic proteins.

In some embodiments, the heterologous luminogenic ER stress response reporter construct used in the methods comprises an ER stress response regulatory sequence in operative association with a sequence encoding a luminogenic reporter molecule. In some embodiments, the ER stress response regulatory sequence comprises a sequence, e.g., X-box-binding protein 1 (XBP1) which allows in-frame transcription or translation of a down stream luminogenic reporter molecule in response to an ER stress. In some embodiments, the construct may further comprise enhancer elements, such as viral enhancer elements, such as a cytomegalovirus (CMV)-derived enhancer element. In some embodiments, the construct comprises a heterologous promoter sequence, e.g., constitutive or regulatory promoter. In some embodiments, the heterologous promoter sequence is a CMV promoter or a β-actin promoter sequence, such as a chicken β-actin promoter sequence.

In some embodiments, the luminogenic reporter is coexpressed with a hypoxic marker chosen from CA-9, SPP1, TXNDC5, RETNLB, ANGPTL1, VEGF, GRP78, GRP94, HIF-1, HIF-2, MMP9, OPN, PAI-1, and OS-9).

In some embodiments, the luminogenic reporter (molecule is chosen from aequorin, an aequorin-fluorescent protein fusion, and a luciferase. In some embodiments, the luminogenic reporter molecule is a luciferase, and in some embodiments, a firefly or renilla luciferase.

In another embodiment, the disclosure provides for compounds identified by any method described above.

Also provided is a transgenic animal. The animal may be a mammal, such as a rat or mouse. In some embodiments, the animal comprises a heterologous luminogenic ER stress response reporter construct disclosed herein. In some embodiments, the reporter is integrated in the genome of the transgenic animal's germ cells.

In some embodiments, the transgenic animal further comprises a mutation associated with a disease characterized by ER stress response activity. In some embodiments, the disease characterized by ER stress response activity is one or more diseases chosen from cancer, diabetes, cardiovascular disease, Alzheimer's disease, Parkinson's disease, amyotrophic lateral sclerosis, polyglutamine disease, prion disease, stroke, bipolar disease, atherosclerosis, wound healing, aging, arthritis, and autoimmune disease. In some embodiments, the cancer is pancreatic cancer, breast cancer, colorectal cancer, skin cancer, brain cancer, lung cancer, stomach cancer, esophageal cancer, bone cancer, colon cancer, uterine cancer, liver cancer, lymphoid cancer, ovarian cancer, rectal cancer, or thyroid cancer. In some embodiments, the cancer is pancreatic cancer, breast cancer, or colorectal cancer. In some embodiments, the mutation is a dominant-negative mutation. In some embodiments, the mutation is a gain-of-function. In some embodiments, the mutation is a loss-of-function. In some embodiments, the mutation is a loss-of-function in a gene chosen from apoE, leptin receptor, leptin, Smad4/DPCA4, and Trp53. In some embodiments, the mutation is K-ras (G12D).

Also provided is a method of detecting ER stress response. In some embodiments, the method comprises exposing a transgenic animal to conditions suspected of modulating the ER stress response and detecting modulation of expression of the luminogenic reporter molecule in at least one cell. In some embodiments, the transgenic animal comprises a heterologous luminogenic ER stress response reporter construct. In some embodiments, the reporter construct comprises an X-box-binding protein 1 (XBP1) regulatory sequence in operative association with a sequence encoding a luminogenic reporter molecule. In some embodiments, the reporter construct is integrated in the genome of the animal's germ cells. In some embodiments, the transgenic animal further comprises a mutation associated with a disease characterized by ER stress response activity.

Also provided is a method of screening a molecule for ER stress response-modulating activity. In some embodiments, the method comprises administering to a transgenic animal a test molecule suspected of having ER stress response-modulating activity and detecting expression of the luminogenic reporter molecule. In some embodiments, a change in luminogenic reporter activity in the animal treated with the test molecule, relative to an untreated control animal, is indicative of the test molecule having ER stress response-modulating activity. In some embodiments, the transgenic animal comprises a heterologous luminogenic ER stress response reporter construct. In some embodiments, the reporter construct comprises an X-box-binding protein 1 (XBP1) regulatory sequence in operative association with a sequence encoding a luminogenic reporter molecule. In some embodiments, the reporter construct is integrated in the genome of the animal's germ cells. In some embodiments, the transgenic animal further comprises a mutation associated with a disease characterized by ER stress response activity.

Also provided is a method of treating at least one disease characterized by ER stress response activity. In some embodiments, the method comprises administering to a patient a therapeutically effective amount of at least one molecule having ER stress response-modulating activity.

In some embodiments, the molecule having ER stress response-modulating activity is screened by a method comprising incubating a transgenic eukaryotic cell with a test molecule suspected of having ER stress response-modulating activity and detecting expression of the luminogenic reporter molecule, wherein a change in luminogenic reporter activity in the cell incubated with the test molecule, relative to an untreated control cell, is indicative of the test molecule having ER stress response-modulating activity.

In some embodiments, the molecule having ER stress response modulating activity is screened by a method comprising incubating a transgenic eukaryotic cell with a test molecule suspected of having ER stress response-modulating activity and a reference molecule and detecting expression of the luminogenic reporter molecule. In some embodiments, a change in luminogenic reporter activity in the cell incubated with the test molecule and the reference molecule, relative to a reference molecule treated control cell, is indicative of the test molecule having ER stress response-modulating activity. In some embodiments, the transgenic cell comprises a heterologous luminogenic ER stress response reporter construct capable of directing expression of a luminogenic reporter molecule in response to an ER stress.

In some embodiments, the molecule having ER stress modulating activity is screened by a method comprising administering to a transgenic animal a test molecule suspected of having ER stress response-modulating activity and detecting expression of the luminogenic reporter molecule. In some embodiments, the transgenic animal comprises a heterologous luminogenic ER stress response reporter construct. In some embodiments, the reporter construct comprises an X-box-binding protein 1 (XBP1) regulatory sequence in operative association with a sequence encoding a luminogenic reporter molecule. In some embodiments, the reporter construct is integrated in the genome of the animal's germ cells. In some embodiments, a change in luminogenic reporter activity in the animal treated with the test molecule, relative to an untreated control animal, is indicative of the test molecule having ER stress response-modulating activity.

Also provided is a method of detecting the toxicity of a molecule suspected of having ER stress response-modulating activity. In some embodiments, the method comprises administering to a transgenic animal a test molecule suspected of modulating ER stress response and detecting expression of the luminogenic reporter molecule to thereby detect the toxicity of the molecule. In some embodiments, the transgenic animal comprises a heterologous luminogenic ER stress response reporter construct. In some embodiments, the reporter construct comprises an X-box-binding protein 1 (XBP1) regulatory sequence in operative association with a sequence encoding a luminogenic reporter molecule. In some embodiments, the reporter construct is integrated in the genome of the animal's germ cells. In some embodiments, the transgenic animal further comprises a mutation associated with a disease characterized by ER stress response activity.

Also provided is a method of estimating the therapeutic ratio of a molecule suspected of having ER stress response activity. In some embodiments, the method comprises administering to a transgenic animal a test molecule suspected of modulating ER stress response, detecting the therapeutic effect of the test molecule; and detecting expression of the luminogenic reporter molecule, wherein the expression of the luminogenic reporter molecule is indicative of the toxicity of the test molecule, to thereby estimate the therapeutic ratio of the test molecule. In some embodiments, the transgenic animal comprises a heterologous luminogenic ER stress response reporter construct. In some embodiments, the reporter construct comprises an X-box-binding protein 1 (XBP1) regulatory sequence in operative association with a sequence encoding a luminogenic reporter molecule. In some embodiments, the reporter construct is integrated in the genome of the animal's germ cells. In some embodiments, the transgenic animal further comprises a mutation associated with a disease characterized by ER stress response activity.

### BRIEF DESCRIPTION OF THE DRAWINGS

FIG. 1 (A) Upper Panel: is a schematic representation of the XBP1-luc reporter construct. Lower panel: is a bar graph of in vitro luciferase activity from different reporter constructs in response to different stresses. HT 1080 cells expressing either a XBP1-luc construct, or an ATF4 or CMV-driven luciferase gene were cultured in 10 μg/ml tunicamycin, glucose depletion, or severe hypoxia (<0.02% O₂) for 24 hours to induce ER stress. (B) is a graphical representation of the incidence of breast carcinomas in Tag mice and Tag-Luc double transgenic mice. (C) Upper panel: shows photographs indicating breast tumors along the mammary chain (arrows) in Tag-Luc and Tag mice. Middle panel: shows photographs of Luc, FVB, tumor bearing Tag-Luc, tumor bearing Tag and non-tumor bearing Tag-Luc mice. Tumor areas are outlined. Lower panel: are overlays of in vivo bioluminescence on the pictures in the middle panel. (D) is a series of fluorescent micrographs of tissue sections from tumors from Tag-Luc and Tag mice. Frozen sections were stained with antiluciferase and anti-XBP1 (top left two panels), anti-luciferase and anti-BiP (bottom left two panels), anti-luciferase and anti-eIF2α (top right two panels), or the control secondary antibodies (bottom right two panels).

FIG. 2 (A) Left panel: is a bar graph of tumor in vivo bioluminesence, normalized to surrounding skin. Middle panel: Is a bar graph of in vitro luciferase activity of mammary tumors normalized to autologous liver. Data in the left and middle panels are represented as the mean±SEM. Right panel: Is a plot of in vitro luciferase activity against in vivo bioluminesence for individual tumors. (B) Left panel: are photographs with overlays of bioluminescence from mammary tumors in Tag-Luc and Tag mice. Right panel: is a bar graph of the in vitro luciferase activity of the indicated tumor from the left panel of FIG. 2 (B). * indicates p<0.05 by Student's t test. (C) is a bar graph of the in vitro luciferase activity of multiple tumors from individual mice. Chart legend in FIG. 2(C) is the same as in FIG. 2(B) (right panel).

FIG. 3 (A) is a bar graph of in vitro luciferase activity of primary tumors from Tag-Luc mice with “Hi” or “Lo” in vivo bioluminesence or tumors from Tag mice after 7-10 days of in vitro culture. Luciferase activity was also measured following tunicamycin treatment. (B) Left panels: are photographs of Tag-Luc mice bearing tumors with synchronous Hi (dark outline in the upper panel) and Lo (light outlines) in vivo bioluminescence (overlays, lower panel). Right panels: are photographs, including bioluminescent overlay, of transplanted Hi (dark circle/arrow; upper left flank) and Lo (light circles/arrows) primary tumors from Tag-Luc mice 14 days after subcutaneously transplantation into four separate quadrants on the dorsal surface of syngeneic FVB mice. (C) is a bar graph of in vivo bioluminesence of primary and transplanted Hi and Lo tumors. Results are the average of four independent experiments.

FIG. 4 (A) shows bioluminescent overlays (top panels) of Tag-Luc mice bearing primary tumors (left panels) or SCID mice bearing transplants (right panels) of the same tumor. Mice were injected with fluorescently-labeled 2-DG or RGD peptide or dye alone and imaged 24 hours later for in vivo bioluminescence and 2-DG, RGD peptide, and dye uptake. Dark arrows indicate tumors with high in vivo bioluminescence and light arrows indicate tumors with low in vivo bioluminescence. (B) is a plot of 2-DG signal against in vivo bioluminescence for the mice imaged in FIG. 4(A). (C) is a bar graph of in vivo bioluminescence in autochthonous tumors after 24 hours of glucose deprivation. (D) Left panel: are photographs with overlays of in vivo bioluminescence of Tag-Luc mice at 0 and 12 hours after treatment with 2 mg of 2-DG or vehicle i.p. Arrows indicate palpable tumors. Right panel: is a bar graph of the average fold increase of in vivo tumor bioluminescence from the left panels of (D). Data are represented as the mean±SEM.

FIG. 5 (A) shows fluorescent micrographs of frozen sections of tumors from Tag-Luc and Tag mice stained with anti-luciferase and anti-CA-IX antibodies. (B) is a bar graph of in vitro XBP1-luc activity in preparations of spontaneous primary tumors from Tag and Tag-Luc mice cultured under normoxic or hypoxic conditions for 48 h. (C) Left panel: are photographs with overlays of in vivo bioluminescence in Tag-Luc mice treated with vehicle or 50 mg/kg DCA (dichloroacetic acid) b.i.d. for 2 days. Bioluminescence was measured after 32 hours. Right panel: is a bar graph of in vivo bioluminescence as shown in the left panel of FIG. 5C and is the average of two experiments. (D) is a bar graph of in vitro luciferase activity in HT1080 cells containing CMV, XBP1, or ATF4-luciferase reporters after treatment with 2-DG, DCA, or glucose deprivation. Luciferase activity was measured after 24 hours.

FIG. 6 (A) is a bar graph of in vitro doubling time and luciferase activity of tumors in the top 15% or bottom 15% of XBP1-luc activity from Tag-Luc mice. (B) is a plot of the fold change of in vivo bioluminescence from tumors in Tag-Luc mice over time. (C) is a plot of the maximum change in vitro XBP1-luc signal against doubling time for tumors from Tag-Luc mice cultured in vitro. (D) is a plot of in vivo bioluminescence plotted against tumor volume for tumors from Tag-Luc mice.

FIG. 7 (A) shows electrophoresed PCR products amplified from genomic DNA from Luc, Tag, or Tag-Luc mice. (B) Electrophoresed XBP1 RTPCR products generated from mRNA isolated from skin fibroblasts of FVB, Tag, Luc, or Tag-Luc mice cultured under normoxic (N) or anoxic (A) conditions for 24-48 hours. To resolve the spliced (S) and unspliced (U) isoforms of the XBP1 transcript, PCR products were digested with Pst I, which cuts only the unspliced isoform. (C) Electrophoresed luciferase RTPCR products generated from mRNA isolated from skin fibroblasts of FVB, Tag, Luc, or Tag-Luc mice cultured under normoxic (N) or anoxic (A) conditions for 48 hours. (D) is a bar graph of in vivo luciferase activity of skin fibroblasts cultured under different conditions. Data is represented as the mean±SEM.

FIG. 8 is a bar graph of in vitro luciferase activity in skin fibroblasts isolated from XBP1-luc, FVB, Tag-Luc and Tag mice and cultured under increasingly severe hypoxic conditions (pO₂ 20%, 2%, 0.5%, and <0.1%). Data is represented as the mean±SEM.

FIG. 9 is a plot of fluorescently-labeled RGD in primary tumors against in vivo bioluminesence for the mice imaged in FIG. 4(A).

FIG. 10 (A) is a graphical representation of the incidence of breast carcinomas in Her2 and Her2-Luc double transgenic mice. (B) shows photographs with bioluminescent overlays (lower panels) of Her2 or Her2-Luc transgenic mice. Breast tumors are outlined in the upper panels. (C) is a bar graph of in vivo bioluminescence in Tag, Tag-Luc, Her2 and Her2-Luc transgenic mice normalized to surrounding skin. (D) is a bar graph of in vitro luciferase activity of mammary tumors from Tag-Luc or Her2-Luc mice normalized to autologous liver. Data are represented as the mean±SEM.

FIG. 11 (A) shows photographs of wounds in a mouse treated with vehicle, DMSO, or a mouse treated with Compound X. (B) is a graph showing the extent of wound healing in mice treated with either vehicle, DMSO or Compound X over time.

FIG. 12 is a chart showing the blood glucose levels in mice fed a high fat diet or control mice in a glucose tolerance test.

FIG. 13 is a graph showing the XBP-1-luc activity in the hearts of mice fed a high fat diet or of control mice.

FIG. 14 (A) shows in vivo modulation of the ER response by compounds. Mice were injected with a control vehicle, Compound X, or Compound Y, and imaged in vivo for bioluminescence initially and 16 hours after treatment. (B) is a graphical representation of the data in 14(A). For each condition, the left bar is time 0 and the right bar is 16 hours.

As used in the present specification, the following words, phrases, and symbols are generally intended to have the meanings set forth below, except to the extent that the context in which they are used indicates otherwise.

“Heterologous luminogenic ER stress response reporter construct” refers to a heterologous construct comprising an ER stress response regulatory sequence in operative association with a sequence encoding a luminogenic reporter molecule, where the reporter is capable of directing expression of the luminogenic reporter molecule under conditions of an ER stress. “ER stress response regulatory sequence” refers to a nucleic acid molecule comprising one or more sequences that allow the in-frame transcription and/or translation of a gene product of a second downstream nucleic acid under conditions of an ER stress. In some embodiments, the second sequence is a sequence encoding a luminogenic reporter molecule.

Two sequences, such as an ER stress response regulatory sequence and a reporter sequence are in “operative association” when the proper expression, e.g., transcription and/or translation of one sequence is dependent on or in response to certain responsive events that have occurred in the other sequence. In some embodiments, an ER stress response regulatory sequence and a reporter sequence are in “operative association” when the reporter sequence is expressed, e.g., transcribed in frame or translated in response to the occurrence of certain ER stress response to the ER stress response regulatory sequence. In some other embodiments, modification of the regulatory sequence in response to an ER stress allows the in-frame transcription or translation of the second downstream nucleic acid. In some embodiments, two sequences in operative association may also be associated with additional sequence elements such as enhancers or promoter sequences. Additional elements may be necessary to drive translation of the reporter construct, such as a Kozak sequence (e.g., a consensus of (gcc)gccRccAUGG, where AUG is the initiation codon). In some embodiments, post-transcriptional modification, e.g., splicing of the ER stress response regulatory sequence in response to an ER stress allows the expression, e.g., in-frame transcription or translation of the second nucleic acid, e.g., a downstream reporter sequence.

One example of a post-transcriptionally regulated ER stress response regulatory sequence is a X-box binding protein 1 (XBP1) regulatory sequence. Accordingly, in some embodiments, the ER stress response regulatory sequence is an XBP1 sequence. Table 1 lists the National Center for Biotechnology Information (NCBI) Entrez GeneID of XBP1 from several species. These GeneIDs may be used to retrieve publicly-available annotated mRNA or protein sequences from the NCBI website, for example, at the following uniform resource locator (URL): http(dot)(slash shash)www(dot)ncbi.nlm.nih.gov/sites/entrez?db=gene.

TABLE 1
XBP from different organisms

| Organism | GeneID |
|---|---|
| Bos taurus | 541236 |
| Caenorhabditis elegans | 175541 |
| Danio rerio | 140614 |
| Drosophila melanogaster | 44226 |
| Gallus gallus | 416918 |
| Homo sapiens | 7494 |
| Mus musculus | 22433 |
| Pan troglodytes | 458735 |
| Rattus norvegicus | 289754 |
| Takifugu rubripes | 654272 |
| Xenopus (Silurana) tropicalis | 407858 |
| Xenopus laevis | 380215 |

For example, using the human GeneID, the unspliced and spliced variant mRNAs for human XBP1 can be obtained. SEQ ID NOs: 1 and 3 are reference sequences NM_{—}005080.3 and NM_{—}001079539.1, which are the unspliced and spliced variants of human XBP1, respectively. The primary translation product of these sequences are provided as SEQ ID NOs: 2 and 4, respectively, which are the reference sequences NP_{—}005071.2 and NP_{—}001073007.1. The spliced mRNA variant lacks a 26 nt intronic sequence, GCAGCACTCAGACTACGTGCACCTCT, which corresponds to nucleotides 541 to 566 of SEQ ID NO:1. Although not relied upon, it is believed that this 26 nucleotide sequence is excised from a constitutively expressed XBP1 transcript in an IRE1 (also known as ERN1, human GeneID 2081) dependent process that results in a frameshift, thereby bypassing an inframe stop codon to produce a full-length, functional translation product in response to ER stress. This full-length XBP1, in turn, acts as a transcription factor that drives the expression of additional downstream genes involved in the ER stress response. In embodiments where the ER stress response regulatory sequence is an XBP1 sequence, the ER stress response regulatory sequence in the ER stress response reporter construct includes a sequence corresponding to the 26 nucleotides excised in the human XBP1 mRNA in response to ER stress. Sequences from other species that correspond to the 26 nucleotides excised from the human XBP1 transcript can be readily identified by the skilled artisan using means known in the art, such as literature annotations, pairwise and/or multiple sequence alignments, dot-matrix, and dynamic programming and known transcript splicing sequences. Additional programs for sequence alignments and comparisons include FASTA (Lipman and Pearson, Science, 227: 1435-41 (1985) and Lipman and Pearson, PNAS, 85: 2444-48), BLAST (McGinnis & Madden, Nucleic Acids Res., 32:W20-W25 (2004) (current BLAST reference, describing, inter alia, MegaBlast); Zhang et al., J. Comput. Biol., 7(1-2):203-14 (2000) (describing the “greedy algorithm” implemented in MegaBlast); Altschul et al., J. Mol. Biol., 215:403-410 (1990) (original BLAST publication)); Needleman-Wunsch (Needleman and Wunsch, J. Mol. Bio., 48 (3): 443-53 (1970)); Sellers (Sellers, Bull. Math. Biol., 46:501-14 (1984); and Smith-Waterman (Smith and Waterman, J. Mol. Bio., 147:195-197 (1981)), and other algorithms (including those described in Gerhard et al., Genome Res., 14(10b):2121-27 (2004)).

In some embodiments, an XBP1 ER stress response regulatory sequence comprises a sequence 1) at least 60, 70, 80, 85, 90, 95, 99, or 100% identical to SEQ ID NO:1 over a sequence of at least 26, 30, 40, 50, 60, 70, 80, 100, 200, 30, 400, 500, 600, or more consecutive nucleotides of SEQ ID NO:1 and 2) including a sequence corresponding to the 26 nucleotide excised in the human XBP1 mRNA in response to ER stress. In some embodiments, the XBP1 ER stress response regulatory sequence comprises all or part of a sequence identical or homologous to a sequence starting at a sequence corresponding to about nucleotide 1, 10, 20, 50, 100, 200, 300, 400, or 500 to a sequence ending at a sequence corresponding to about nucleotide 566, 560, 570, 580, 590, 600, 620, 640, 650, 660, 670, 671, 680, 700, 750, or 800 of SEQ ID NO:1. “Sequence corresponding to” refers to a sequence having a percent identity over a sequence of the length specified above, relative to SEQ ID NO:1, as identified by sequence alignment. In some embodiments, the XBP1 ER stress response regulatory sequence comprises a sequence at least 80, 85, 90, or 95% identical to a fragment of at least 100, 200, 400, or 500 consecutive nucleotides of nucleotides 1-671 of SEQ ID NO:1, wherein the fragment comprises a sequence corresponding to nucleotides 541 to 566 of SEQ ID NO:1.

Alternative ER stress response regulatory sequences may be used in the methods disclosed herein. In some embodiments, an ER stress response regulatory sequence can be a sequence activating the expression, e.g., in-frame transcription or translation of the reporter molecule in response to an ER stress or a factor activated by an ER stress, e.g., the activated XBP1 or genes or factors activated by XBP1. For example, spliced, activated XBP1 is a transcription factor that drives the transcription and/or translation of additional genes. Accordingly, in some embodiments, nucleic acids comprising XBP1 binding sites and/or genes (including promoters) that are upregulated in response to ER stress in an XBP1-dependent manner can be ER stress response regulatory sequences for use in the disclosed methods. In some embodiments, an ER stress response regulatory sequence comprises one or more copies of the XBP1-binding sequence GATGACGTG(T/G)NNN(A/T)T and/or the highly conserved ACGT subsequence; for example, 1, 2, 3, 4, 5, 6, 7, or more copies of these sequences.

Some of the downstream transcriptional targets of XBP1 have been identified. Accordingly, the regulatory sequences of these genes, as well as the conserved binding sequences of these genes can be used as ER stress response regulatory sequences in the heterologous luminogenic ER stress response reporter constructs provided herein. Thus, in some embodiments, ER stress response regulatory sequences include one or more of the sequences GCCACG (CCACG-box), CGACGTGG (UPRE A), GTGACGTG (UPRE B), G(C/T)(C/G)ACGT (ACGT core), CCAATC (CAAT box), and CGGAAG (ETS domain), including combinations thereof and sequence variants, including, e.g., 1, 2, or 3 nucleotide mismatches to each of these sequence. In some embodiments, an ER stress response regulatory sequence comprises at least 1, 2, 3, 4, 5, 6, or more copies of one or more of these sequences, e.g., at least 1, 2, 3, 4, 5 or all 6 sequences.

In some embodiments, the regulatory elements of one or more genes regulated by XBP1 can be used as an ER stress response regulatory sequence. In some embodiments, the ER stress response regulatory sequence comprises a sequence from a gene chosen from one or more of ERdj4 (a.k.a. DNAJB9, human GeneID 4189), p58^{IPK} (a.k.a. DNAJC3, human GeneID 5611), EDEM (human GeneID 9695), PD1-P5 (a.k.a. PDIA6, human GeneID 10130), RAMP4 (a.k.a. SERP1, human GeneID 27230), HEDJ (a.k.a. DNAJB11, human GeneID 51726), and BiP (a.k.a. HSPA5, human GeneID 3309). In some embodiments, the ER stress response regulatory sequence comprises a sequence from ATF4 (mouse GeneID 11911, mouse mRNA NM_{—}009716.2; human GeneID 468, human mRNAs NM_{—}001675.2 and NM_{—}182810.1). Additional XBP1 targets include the following mouse GeneIDs: 11911 (ATF4), 12317, 12330, 13198, 67819, 70377, 83945, 67838, 27362, 66861, 192193, 108687, 13666, 50527, 67475, 109815, 64209, 22027, 14828, 18453, 14827, 72599, 71853, 108954, 114679, 93684, 81500, 106200, 76299, 22230, 320011, and 269523. Additional mouse sequences identified as XBP1 targets are known in the art and can be used in the methods described herein. ER stress response regulatory sequences, such as those comprising ACGT and/or CCACG elements, generally occur within the first 600, 500, 400, 300, 200, or 100 nucleotides of the transcription start site of the XBP1-responsive genes described above. In some embodiments, ER stress response regulatory sequences comprise combinations of sequence from different XBP1-regulated genes and sequence variants thereof, including, e.g., sequences with 70, 80, 90, 95, or 99% identity over at least 30, 40, 50, 60, 70, 80, 100, 200, 30, 400, 500, 600, or more consecutive nucleotides proximate to the transcription start site of these genes, e.g., within approximately 600-200 nucleotides of transcription start. In some embodiments, an ER stress response regulatory sequence comprises sequences from 1, 2, 3, 4, 5, 6, or more of one or more of the sequences listed above. Sequences for the genes listed above may be from any vertebrate including, for example, human, mouse, rat, cow, pig, macaque, chimp, and frog. The aforementioned human and mouse GeneIDs are exemplary only. Sequences for these genes from additional organisms will be readily identified based on the reference annotations, above.

Any suitable expression vector may be used to provide the ER stress response reporter constructs provided herein. Vectors may be modified by methods known in the art to be suitable for a particular application, such as generation of a transgenic animal. In some embodiments, the ER stress response reporter construct contains a constitutive promoter operatively linked to the ER stress regulatory sequence, e.g., in operative association with a reporter molecule. In some other embodiments, the ER stress response reporter construct contains an inducible promoter operatively linked to the ER stress regulatory sequence. In one embodiment, a heterologous luminogenic ER stress response reporter construct is generated in the pEGFP-N1 vector (reference accession no. GI:1377911; where nucleotides 1-589 correspond to the human cytomegalovirus (CMV) immediate early promoter, nucleotides 59-465 correspond to enhancer region, nucleotides 554-560 correspond to the TATA box, nucleotide 583 corresponds to the transcription start point, and nucleotides 1552-1557 and 1581-1586 correspond to SV40 polyadenylation signals). Additional sequences may be used to enhance the transcription of the ER stress response reporter constructs. In some embodiments, an ER stress response reporter construct further comprises a heterologous enhancer sequence, such as a viral enhancer sequence, derived from a virus such as SV40, CMV, HTLV, MLV, or MSV. In some embodiments, the viral enhancer is from CMV. In some embodiments, the construct further comprises a transcription start site-proximate sequence from the chicken β-actin gene (GeneID 396526).

“Luminogenic reporter molecule” refers to a product of translation that, when expressed, can emit photons in the absence of excitatory photons. In some embodiments, the luminogenic reporter molecule is an aequorin (such as from Aequoria victoria, accession number GI:461375), an aequorin-fluorescent protein fusion or a luciferase, such as a firefly luciferase (see, for example, accession numbers GI:160794, GI:3929282, and GI:198409931), a renilla luciferase (see, for example, accession numbers GI:160820 and GI:1246926), metridia luciferase (see, for example, accession numbers GI:189547892 and GI:189547890), or bacterial luciferase (see, for example, accession numbers GI:294817230, GI:294817074, NP_{—}792987.1, NP_{—}792986.1, and NP_{—}660074.1). In some embodiments, the luminogenic reporter molecule is a luciferase and in some embodiments, a firefly luciferase (Feldman et al., Mol Cancer Res., 3: 597-605 (2005)).

Luminogenic reporter molecules may vary in their requirements for additional molecules, such as cofactors, needed to emit photons. For example, aequorin is sensitive to Ca²⁺ concentrations and requires coelenterazine or coelenterazine analogs (including, for example, h-coelenterazine, f-coelenterazine, cp-coelenterazine, and hcp-coelenterazine, and others known in the art). Firefly luciferase requires oxygen, luciferin or its analogs (including aminoluciferin), ATP and magnesium, while renilla luciferase requires coelenterazine or its analogs. Bacterial luciferases typically require reduction of a flavin mononucleotide group and oxygen as an activator. Accordingly, based on the present disclosure and knowledge in the art, the skilled artisan will understand that molecules that enable the luminogenic reporter molecule to emit photons will need to be provided in the methods described herein, e.g., in culture medium for in vitro methods or in a transgenic animal for in vivo methods.

In some embodiments, the methods disclosed herein may include the step of incubating a cell comprising a heterologous luminogenic ER stress response reporter construct in conditions suspected of modulating the ER stress response. Cells for use are eukaryotic and can be used in vivo or in vitro. The cells may be obtained from a variety of sources including established cell lines or primary cells. The cells may be from, for example, humans or other animals—such as livestock, domestic, and wild animals. In some embodiments, the cells are from an avian, bovine, canine, equine, feline, ovine, pisces/fish, porcine, primate, rodent, or ungulate source. The cells can be from an animal at any stage of development, including adult, youth, fetal, or embryo. In some embodiments, the cell is from a mammal, e.g., a rodent, e.g., a rat or mouse. The cell may be derived from any of the three primordial germ layers—ectoderm, mesoderm, or endoderm. Exemplary cell types include skin, heart, skeletal muscle, smooth muscle, kidney, liver, lung, bone, pancreas, central nervous tissue, peripheral nervous tissue, circulatory tissue, lymphoid tissue, mammary tissue, intestine, spleen, thyroid, connective tissue (e.g., chondrocytes and fibroblasts), or gonad.

In some embodiments, any of the cells described above are from a transgenic animal, such as a mouse, where the transgenic animal contains a heterologous luminogenic ER stress response reporter construct in the genome of its germline cells. “Transgenic animal” refers to a non-human animal, such as a cow, pig, sheep, rabbit, mouse, rat, or non-human primate comprising a mutation in the genome of at least a subset of its germline cells, where the mutation is the result of human genetic engineering, including full or partial gene deletions and insertions of heterologous transgenes. In some embodiments, the transgenic animal is chimeric, heterozygous, or homozygous for the heterologous luminogenic ER stress response reporter construct. In some embodiments, the transgenic animal is heterozygous or homozygous for the heterologous luminogenic ER stress response reporter construct.

To produce transgenic animals, any method for introducing a recombinant construct or transgene into an embryo may be used, including, for example, microinjection, gene gun, transfection, liposome fusion, electroporation, viral infection, and the like. In some embodiments, the method comprises microinjection, which involves injecting a DNA molecule into the male pronucleus of fertilized eggs. The above methods for introducing a recombinant construct/transgene into mammals and their germ cells were originally developed in the mouse. These methods were subsequently adopted for use with larger animals, including livestock species. Microinjection of DNA into the cytoplasm of a zygote can also be used to produce transgenic animals. In some embodiments, transgenic mice can be bred or crossed to one or more lines of transgenic mice to generate mice that contain one or more mutations.

In some embodiments, a transgenic animal comprises a mutation that is associated with a disease characterized by ER stress response activity. A “disease characterized by stress response activity” refers to a disease that is caused by or associated with ER stress response activity. A mutation associated with diseases characterized by ER stress response activity includes, without limitation, loss of function of apoE (mouse GeneID 11816), leptin receptor (mouse GeneID 6847), leptin (mouse GeneID 16846), Smad4/DPCA4 (Izeradjiene et al., Cancer Cell, 11: 229-43 (2007)), and Trp53 (Izeradjiene et al., Cancer Cell, 11: 229-43 (2007)). In some embodiments, leptin receptor deficient mice are db/db mice. In some embodiments, leptin deficient mice are ob/ob mice. Mutations associated with diseases characterized by ER stress response activity include, without limitation, the K-ras mutation, K-ras (G12D) (Hingorani et al., Cancer Cell, 4: 437-450 (2003), Tuveson et al., Cancer Cell, 5: 375-87 (2004), Cook et al., Methods Enzymol. 439: 73-85 (2008), and Izeradjiene et al., Cancer Cell, 11: 229-43 (2007)).

Cells for use in the methods described herein may contain genetic modifications in addition to the heterologous luminogenic ER stress response reporter constructs provided herein. For example, in some embodiments, the cells may include mutations associated with an increased risk of tumorogenic disease, in order to evaluate the ability of a particular treatment, such as a chemotherapeutic agent, to modulate the ER stress response of a cancer cell. “Tumorigenic diseases” refers to genetic changes associated with disregulation of normal cell growth and division control, leading to pre-cancerous, cancerous, or metastatic cell growth. The mutations associated with an increased risk of tumorogenic disease may be naturally occurring, i.e., primary cells containing non-engineered, spontaneous, de novo mutations, or engineered, e.g., established cell lines or primary cells containing one or more engineered mutations including gain of function “oncogenes” (such as ABL1, BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MMTV-PyVT (Tag), MMTVneu (Her2), MYB, MYC, MYCL1, MYCN, NRAS, PIM1, PML, RET, SRC, TAL1, TCL3, and YES) or loss-of-function of a tumor suppressor gene (such as APC, BRCA1, BRCA2, MADH4, MCC, NF1, NF2, RB1, P53, and WT1).

In some embodiments, cells for use in the methods disclosed herein also exhibit altered activity of proteins that modulate apoptosis. The altered activity may be due to genetic modifications (e.g., hypermorphic, hypomorphic, or null alleles), reversible chemical genetics, or altered transcription and/or translation rates. Proteins that modulate apoptosis include anti-apoptotic proteins, such as BCL2 (human GeneID 596) and BCLX_{L} (a.k.a. BCL2L1, human GeneID 598), as well as pro-apoptotic proteins, such as BAX (human GeneID 581) and BAK (a.k.a. BAK1, human GeneID 578). In some embodiments, the cell exhibits increased or decreased activity of one or more proteins that modulates apoptosis. In some embodiments, the cell exhibits increased activity of pro-apoptotic proteins or decreased activity of anti-apoptotic proteins.

Methods are provided for detecting modulation of the ER stress response by detecting a luminogenic reporter molecule in cells comprising a heterologous luminogenic ER stress response reporter construct. Generally, the methods comprise incubating a cell under conditions suspected of modulating the ER stress response. The conditions may be an environmental or physiological treatment, e.g., thermal, barometric, mechanical, or photic stimulus. The condition may also be a chemical treatment, e.g., changes in pO₂ concentration, osmolarity, pH, or a small molecule (e.g. pharmacological) or biological agent, or any combination of the above.

In some embodiments, the cell is incubated under conditions known to modulate the ER stress response, for example, to potentiate the ER stress response in order to evaluate the ability of a test treatment to modulate the ER stress response. For example, in some embodiments, it may be desirable to identify agents that inhibit the ER stress response, since this pathway may help tumor cells to survive harsh microenvironments and inhibiting this survival mechanism may enhance the efficacy of a cytotoxic therapy. Thus, in some embodiments, a cell is incubated in conditions known to activate the ER stress response, and a test treatment is applied to determine if the ER stress response decreases. In some embodiments, the methods described herein can be used to identify conditions that activate ER stress response, since such conditions may be directly cytotoxic. In some embodiments, the cell is incubated with a test molecule and/or a reference molecule. “Reference molecule” refers to a molecule that has a known activity in an assay. In some embodiments, detecting the activity of a test molecule and a reference molecule indicates the potency of the test molecule.

In some embodiments, a molecule having ER stress response-modulating activity may modulate the activity of proteins involved in ER stress response. These proteins involved in ER stress response include, by way of example and without limitation, the protein chaperone glucose-regulated protein (GRP78), inositol-requiring kinase 1 (IRE1α), PRKR-like endoplasmic reticulum kinase (PERK), activating transcription factor 6 (ATF6), and C/EBP homologous protein (CHOP) (Kim et al., Nature Reviews Drug Discovery, 7: 1013-1030 (2008)).

Conditions known to activate the ER stress response include low oxygen (e.g., less than 10, 9, 8, 7, 6, 5, 4, 3, 2, 1, 0.5, 0.1, or 0.01% pO₂), low glucose (e.g., less than 50, 40, 35, 30, 25, 20, 15, 10, or 5 mM glucose), or low pH (e.g., less than 7.0, 6.5, 6.0, 5.5, 5.0, 4.5, 4.0, 3.5, 3.0, 2.5, 2.0, 1.5, or 1.0). Chemicals known to stimulate ER stress response include proteasome inhibitors (such as bortezomib/PS 341, PS-519, lenalidomide, NPI-0052, carfilzomib/PR-171, as well as others known in the art), brefeldin A, beta-mercaptoethanol, tunicamycin, 2-deoxy-d-glucose, dichloroacetic acid, thapsigargin, dithiothreitol. The skilled artisan will understand that the duration of any treatment and/or the concentration of any agent will be modified for the particular cell type and application (e.g., ex vivo or in vivo) so as to produce a detectable change in the ER stress response reporter construct, relative to the cell in the absence of the treatment.

In some embodiments, conditions that activate an ER stress response reporter construct provided herein may also stimulate transcription and/or translation of a hypoxic marker. Hypoxic markers include, for example, carbonic anhydrase 9 (CA9, human GeneID 768), secreted phosphoprotein 1 (SPP1, human GeneID 6696), thioredoxin domain containing 5 (TXNDC5, human GeneID 81567), resistin like beta (RETNLB, human GeneID 84666), angiopoietin-like 1 (ANGPTL1, human GeneID 9068), 78 kDa glucose upregulated protein (GRP78, human GENEID 3309), 94 kDa glucose upregulated protein (GRP94, human GENEID 7184), matrix metallopeptidase 9 (MMP9, human GENEID 4318), osteopontin (OPN, human GENEID 6696), plasminogen activator inhibitor-1 (PAI-1, human GENEID 5054), osteosarcoma amplified 9, endoplasmic reticulum lectin (OS-9, human GENEID 10956), vascular endothelial growth factor (VEGF), hypoxia inducible factor-1 (HIF-1), and hypoxia inducible factor-2 (HIF-2). “VEGF” refers to at least one member of a family of growth factors that includes, for example, VEGFA (human GENEID 7422), VEGFB (human GENEID 7423), VEGFC (human GENEID 7424), and VEGFD (human GENEID 2277). “HIF-1” refers to a complex of HIF1A (human GENEID 3091) and aryl hydrocarbon receptor nuclear translocator (Arnt, human GENEID 405). “HIF-2” refers to a complex of HIF2A (human GENEID 2034) and aryl hydrocarbon receptor nuclear translocator (Arnt, human GENEID 405).

The methods disclosed herein are useful for identifying treatments useful for therapeutic interventions, for example, in treating diseases such as cancer, diabetes, cardiovascular disease, Alzheimer's disease, Parkinson's disease, amyotrophic lateral sclerosis, polyglutamine disease, prion disease, stroke, bipolar disease, atherosclerosis, wound healing, aging, arthritis, and/or autoimmune disease. In some embodiments, the cancer is chosen from pancreatic cancer, breast cancer, colorectal cancer, skin cancer, brain cancer, lung cancer, stomach cancer, esophageal cancer, bone cancer, colon cancer, uterine cancer, liver cancer, lymphoid cancer, ovarian cancer, rectal cancer, and thyroid cancer. In some embodiments, the cancer is chosen from pancreatic cancer, breast cancer, and colorectal cancer. For example, the unfolded protein response and ER stress pathways may be involved in insulin resistance, diabetes, and obesity (Glimcher and Lee, Ann NY Acad. Sci., 1173 Suppl 1:E2-9 (2009). Ozcan et al., Science, 306:457-61 (2004), Yang et al., Cell Metab., 11:467-78. (2010)).

The in vivo methods provided herein, for example, provide the skilled artisan with useful pharmacokinetic data for a particular agent including, inter alia, efficacious doses of agents that modulate the ER stress response pathway. Effective dosages achieved in one animal may be converted for use in another animal, including humans, using conversion factors known in the art. For example, human equivalent dosing (HED) in mg/kg based on animal dosing may be given by the following equation: HED (mg/kg)=animal dose (mg/kg)×(Km^{animal}/Km^{human}), where Km=weight/surface area (kg/m²).

Exemplary conversion factors based on the above equation are shown in the following table.

TABLE 2

From:

| | Mouse | Rat | Monkey | Dog | Human |
|---|---|---|---|---|---|
| To: | (20 g) | (150 g) | (3.5 kg) | (8 kg) | (60 kg) |
| Mouse | 1 | 0.5 | 0.25 | 0.17 | 0.08 |
| Rat | 2 | 1 | 0.5 | 0.25 | 0.14 |
| Monkey | 4 | 2 | 1 | 0.6 | 0.33 |
| Dog | 6 | 4 | 1.7 | 1 | 0.5 |
| Human | 12 | 7 | 3 | 2 | 1 |

In some embodiments, the toxicity or therapeutic ratio of an agent may be evaluated by detecting ER stress response. The “therapeutic ratio” refers to a comparison of the amount of an agent that causes a therapeutic effect to the amount that causes toxicity. The amount of ER stress response may be indicative of the toxicity of an agent.

Cells for use in the methods described herein may be cultured by a variety of means, including either in vivo or ex vivo culture. In vivo culturing may be autogenic (i.e., in the same animal from which the cells are derived; autogenic culturing may, but need not, include isolation, ex vivo culturing, and re-implantation of the cells), allogenic (i.e., in a different animal from which they are derived, but in an animal of the same strain or variety), or syngenic (i.e., in a different animal from which they are derived, but in an animal that is immunogenically compatible with the animal from which the cells are derived). In some embodiments, the cells may be cultured xenogenically (i.e., in a different species).

For in vivo culturing methods luminogenic reporter molecules may be detected non-invasively. “Non-invasively” means a signal above the limit of detection can be detected in a live, intact, whole animal, without the need for surgery, e.g., signal can be detected through the skin. Luminogenic reporter molecules may be detected non-invasively in whole, live animals that are fully alert and moving, partially sedated, or fully anesthetized. In some embodiments, luminogenic reporter molecules are detected in fully anesthetized animals. Luminogenic reporter molecules may be detected with suitable detection systems known in the art. Luminogenic reporter molecules can be detected with various exposure times, including about 0.001, 0.005, 0.01, 0.05, 0.1, 0.5, 1, 5, 10, 20, 40, or 60 seconds, 2, 3, 4, 5, 6, 7, 8, 9, 10, 15, 20, 25, or 30 minutes. Luminogenic reporter molecule signals can be normalized by a variety of means, including fold induction over time before and after a particular treatment, and/or relative to control tissues, such as autologous skin or liver. In some embodiments, the ER stress response is modulated when expression of a luminogenic reporter molecule changes (i.e., increases or decreases) about 10, 20, 30 40, 50, 60, 70, 80, or 90%, or 2, 3, 4, 5, 10, 15, 20, 25, 30, 40, 50, 60, 70, 80, 100, 250, 500, 1000, 5000, or 10000-fold.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

### EXAMPLES

### Example 1

### Generation of Transgenic Mice and In Vivo Imaging of ER Stress Response

1.1 XBP1-Luciferase Reporter

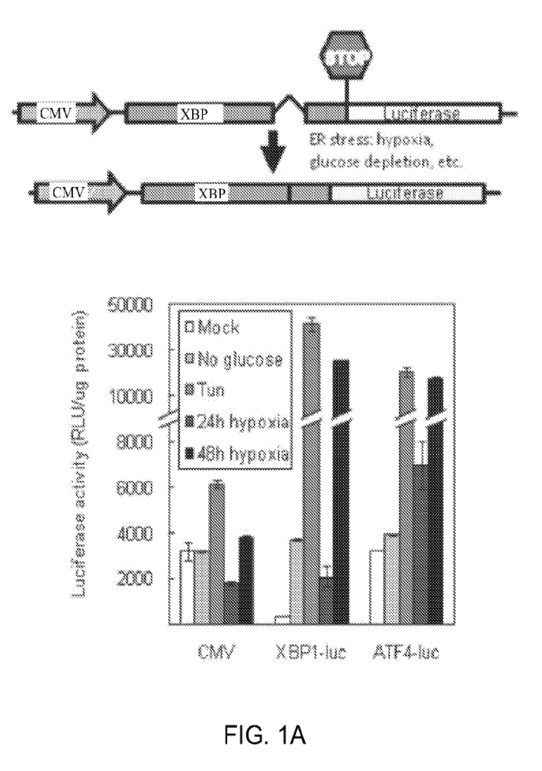

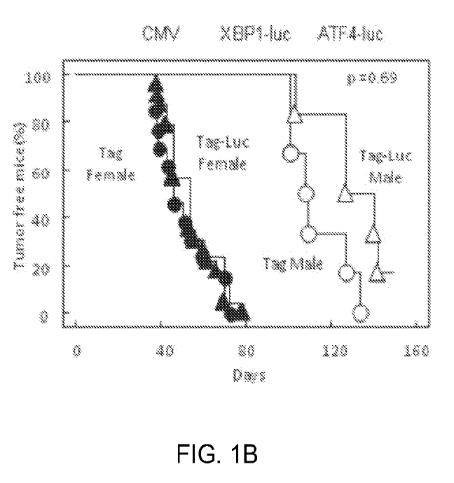

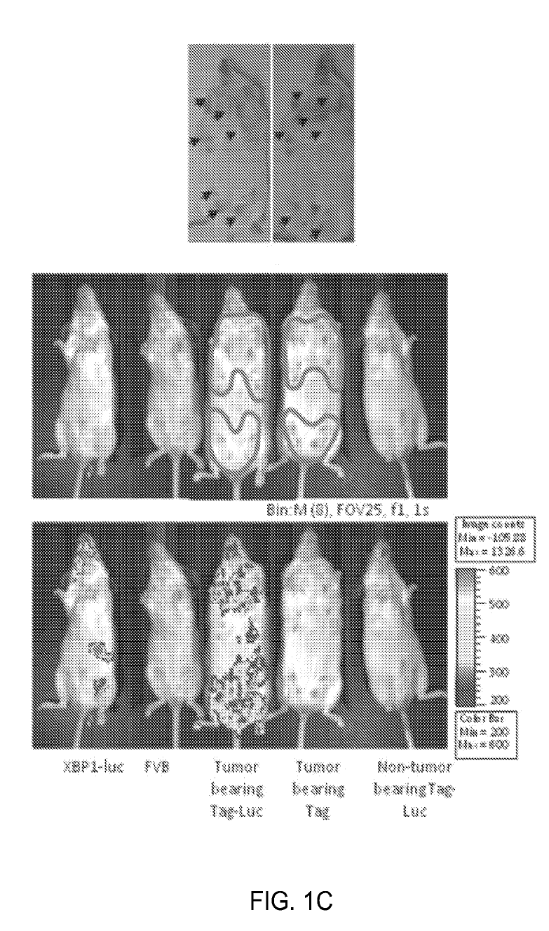

The first 208 amino acids of the unspliced human XBP1-1 was amplified by PCR and subcloned into pEGFP-N1 (Clontech, Mountain View, Calif.). This plasmid was subsequently digested with Bam HI and Not Ito remove the EGFP and a PCR-generated firefly luciferase gene was cloned downstream of the XBP1-1 gene such that the luciferase gene was in frame only in the “spliced” reading frame. Under ER stress conditions, the XBP1-luc transcript is spliced, shifting the stop codon out of frame, allowing expression of the luciferase gene. A graphical representation of this reporter construct is shown in FIG. 1A. At the top of the figure is shown, from left to right, a CMV sequence, in a right-pointed arrow; an XBP sequence in a box, and a “stop” signal in the top construct.

This reporter construct was tested in vitro. The human fibrosarcoma cell line HT1080 was obtained from ATCC (Manassas, Va.). Cells were maintained at 37° C. with 5% CO₂ in DMEM supplemented with 10% FBS and 1% penicillin-streptomycin antibiotics. HT1080 cells (FIG. 1A, lower panel) were stably transfected with the XBP1-Luc construct. Compared to control cells transformed with a CMV-Luc construct, XBP1-Luc cells strongly induced luciferase activity under conditions that increase ER stress, such as tunicamycin treatment, glucose depletion or hypoxia. See FIG. 1A, lower panel. A second UPR reporter, ATF4-Luc, was also induced under conditions that increase ER stress. Thus, these reporters could be used to detect ER stress in vitro. The bars are as indicated in the legend, from left to right.

1.2 Transgenic Mice

FVB/N-Tg(MMTV-PyVT)634 Mul/J (Tag), FVB/N-Tg(MMTVneu)202Mul/J (Her2), FVB/NJ and SCID mice were purchased from The Jackson Laboratory (Bar Harbor, Me.). Mice were housed in sterile cages, fed autoclaved chow and acidified water, and bred at Stanford University Research Animal Facility. Mice were maintained according to Stanford Institutional Care and Use Committee guidelines.

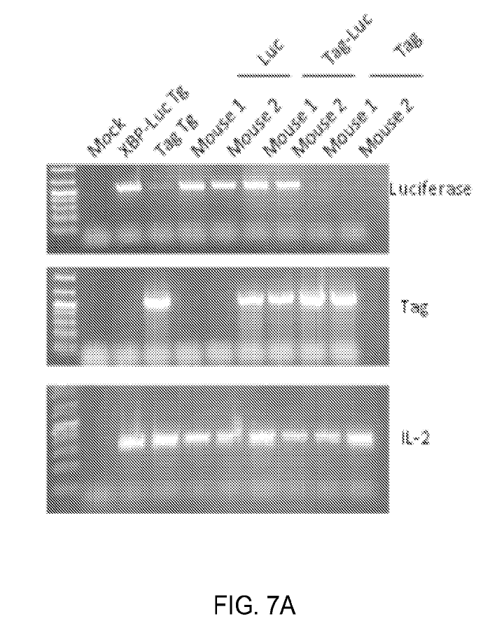

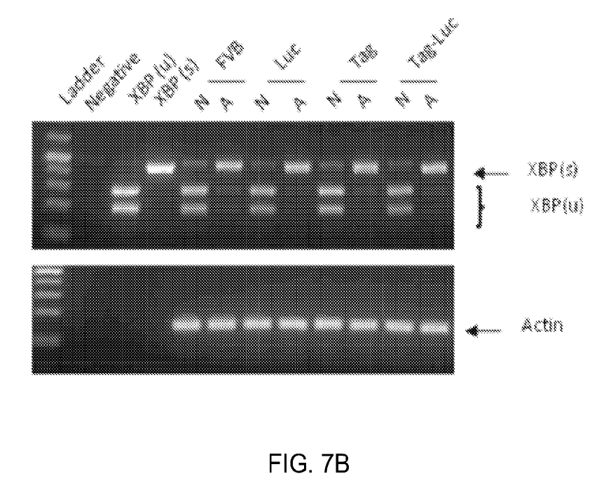

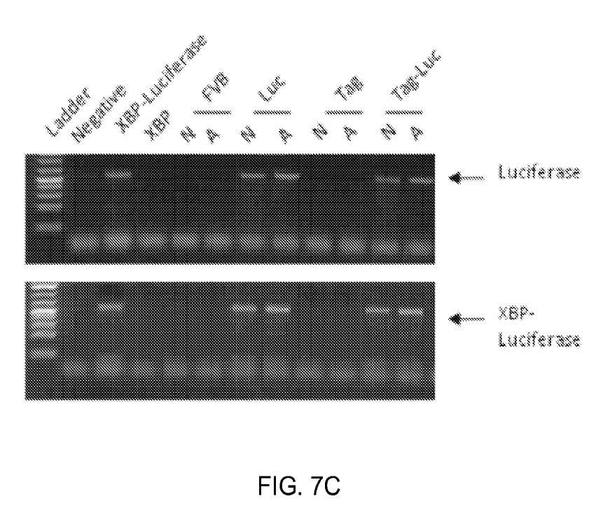

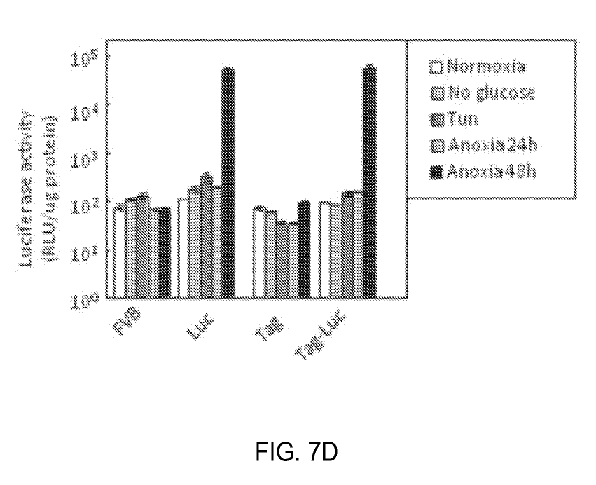

The XBP1-Luc transgene was injected into FVB/N oocytes. Genomic DNA was isolated from the tails of the resulting mice and was screened by PCR for integration of the luciferase gene, and a single positive founder line was maintained. Expression of the XBP1-Luc transgene was detected by rtPCR. Total RNA was isolated from cells using the RNeasy (Qiagen, Valencia, Calif.) RNA-isolation system. cDNA was generated using MMTV reverse transcriptase and random hexamers. The mRNA was detected using the following primers Luc: 5′-agaactgcctgcgtgagatt-3′ and 5′-cacacagttcgcctctttga-3′; Luc-XBP1 5′-aaacagagtagcagctcagactgc-3′ and tccacctcgatatgtgcatc-3′; XBP1 5′-aaacagagtagcagctcagactgc-3′ and 5′-tccttctgggtagacctctgggag-3′. See FIG. 7A. RT-PCR for XBP1 amplified both the unspliced and spliced isoforms in both mouse and human XBP1 sequences. This enabled detection of splicing of the endogenous and transgenic XBP1 mRNA. See FIG. 7C. To resolve the unspliced and spliced isoforms, PCR products were digested with Pst I, which cuts the unspliced XBP1 mRNA into two fragments (290 bp and 183 bp) and leaves the spliced XBP1 mRNA intact (447 bp). See FIG. 7B.

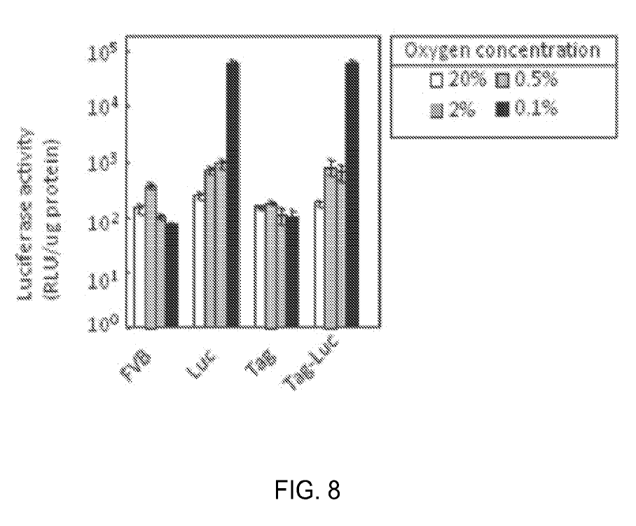

Next, a XBP1-Luc transgenic mouse was bred to breast cancer prone Tag mice, to generate double transgenic Tag x XBP1-luc mice (Tag-Luc mice; FIG. 1B & FIG. 7A). Tag mice express the middle T antigen under the control of the MMTV promoter and develop multiple primary mammary carcinomas at a mean interval of 42 days. Normal skin fibroblasts isolated from Tag-Luc mice constitutively expressed the XBP1-luc fusion mRNA transcript (FIGS. 7B & C) in vitro. Under conditions that induce ER stress, such as glucose deprivation or hypoxia (FIG. 7B), the XBP1-luc transcript was spliced and produced luciferase activity (FIG. 7D and FIG. 8).

### Example 2

### Primary Tumors from Transgenic Mice have Increased XBP1-Luc Activity

Tag-Luc double transgenic mice developed primary tumors at rates similar to control Tag mice (FIG. 1B; for females, p=0.72; for males, p=0.07). Tumor incidence curves were estimated with the Kaplan-Meier method and analyzed with the Cox log-rank method for significance.

In vivo bioluminescence, which corresponds to XBP1-Luc activity, was detected noninvasively using the IVIS imaging system (Caliper LifeSciences, Hopkinton, Mass.). Mice were injected i.p. (intraperitoneally) with luciferin (300 mg/kg dissolved in PBS; Caliper). During imaging acquisition, mice were anesthetized via inhaled 3% isoflurane (Abbott Laboratories Ltd, Abbott Park, Ill.). Exposure time ranged from ten seconds to ten minutes depending on the mouse studied. Image analysis and quantification was performed using Living Image software (Caliper LifeSciences, Hopkinton, Mass.). All images were analyzed with a binning of 10. For comparisons between mice, the bioluminescent signal of the tumors was normalized to adjacent skin.

Imaging of tumor bearing Tag-Luc mice showed that regions corresponding to the mammary tumors exhibited bioluminescent signal (FIG. 1C). By contrast, primary tumors arising in control Tag mice exhibited only background signal. A single transgenic XBP1-luc mouse had basal bioluminescent signal in the spleen, consistent with previous reports of normal XBP1 activity in that organ. Tag-Luc mice without tumors did not have appreciable bioluminescent signal along the mammary chains (FIG. 1C). Thus, using the XBP1-luc reporter, tumors under ER stress could be visualized non-invasively in vivo. The legend in FIG. 1C shows image counts (min=105.88, max=1326.c) and a color bar, min=200, max=600).

### Example 3

### Tumor Histology

Frozen tumor sections from Tag-Luc and Tag mice were stained for luciferase and various ER stress proteins. Tumors were isolated and immediately imbedded in O.C.T. (optimum cutting temperature compound; Sakura Finetek, Torrence, Calif.). Cryosections were blocked with a 5% mixture of goat and donkey anti-serum. Sections were then stained with the respective antibodies according to the manufacturer's instructions. Antibody binding was detected with AF555 labeled goat anti-mouse, AF555 labeled goat anti-rabbit, AF488 labeled donkey anti-goat or AF488 labeled goat anti-rabbit secondary antibodies. Rabbit anti-BiP, Rabbit anti-Phospho eIF2α antibodies were obtained from Cell Signaling (Danvers, Mass.). Rabbit anti-XBP1 antibodies were obtained from Abcam (Cambridge, Mass.). Goat anti-luciferase antibodies were obtained from Promega (Madison, Wis.) and mouse anti-luciferase antibodies were obtained from AdD Serotec (Oxford, UK).

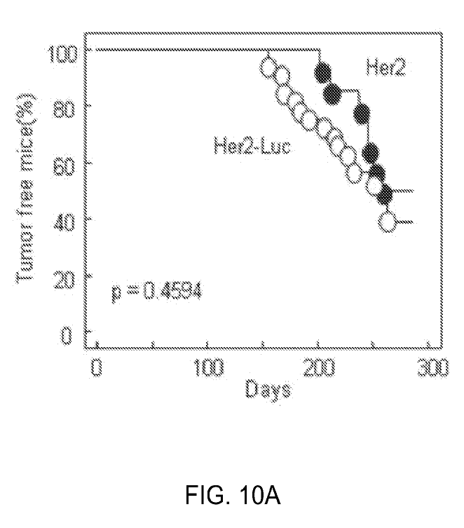

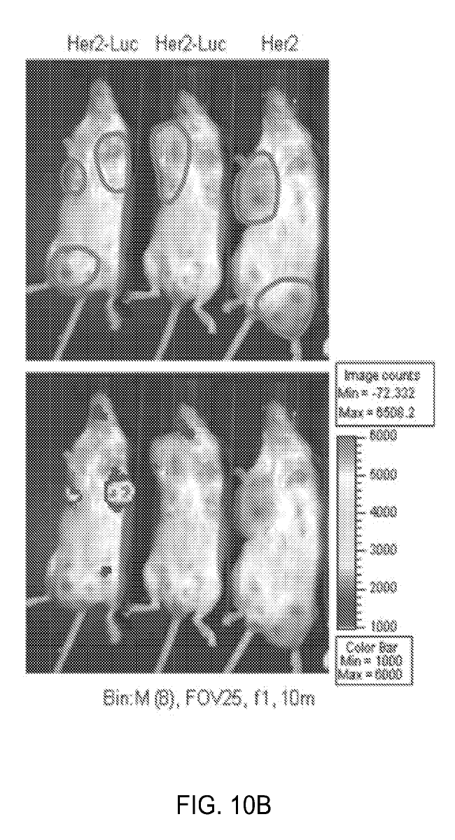

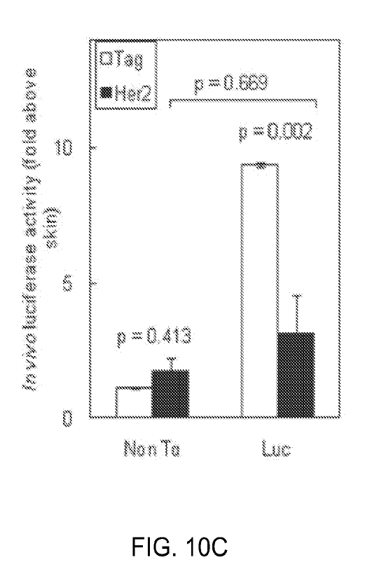

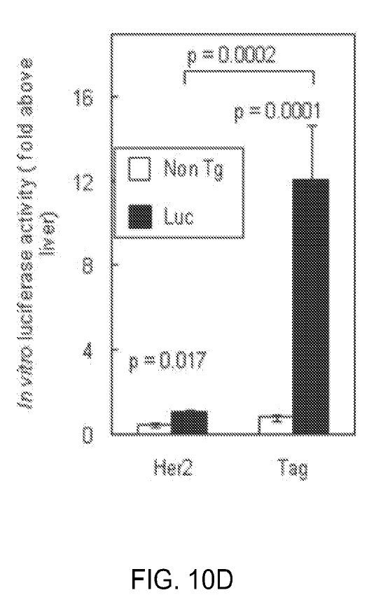

In tumors from Tag-Luc mice, luciferase colocalized with XBP1, as well as other UPR proteins including BiP and eIF2α. In contrast, luciferase was not detected in tumors from Tag mice (FIG. 10). Therefore, primary tumors from Tag-Luc mice exhibited XBP1-luc activity in regions of primary tumors experiencing ER stress.

### Example 4

### Tumors in the Same Mouse have Heterogeneous Bioluminescence, which is Modulated by the Tumor Microenvironment

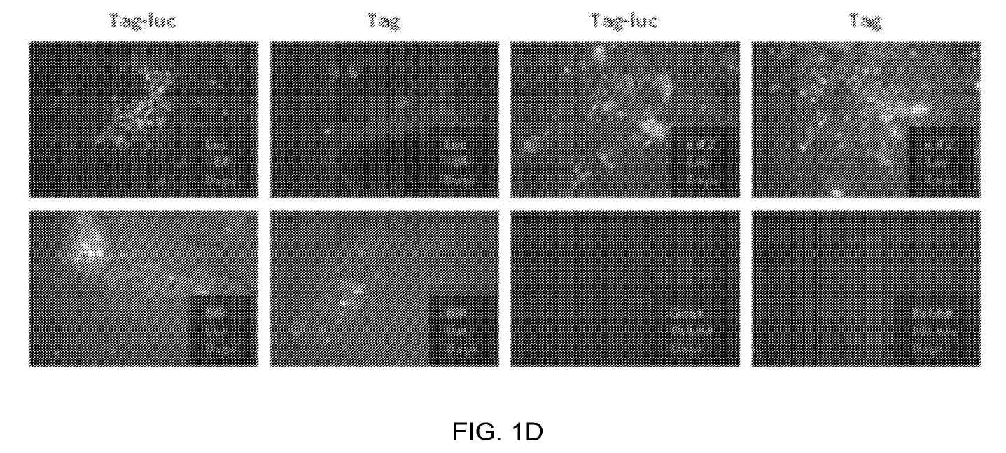

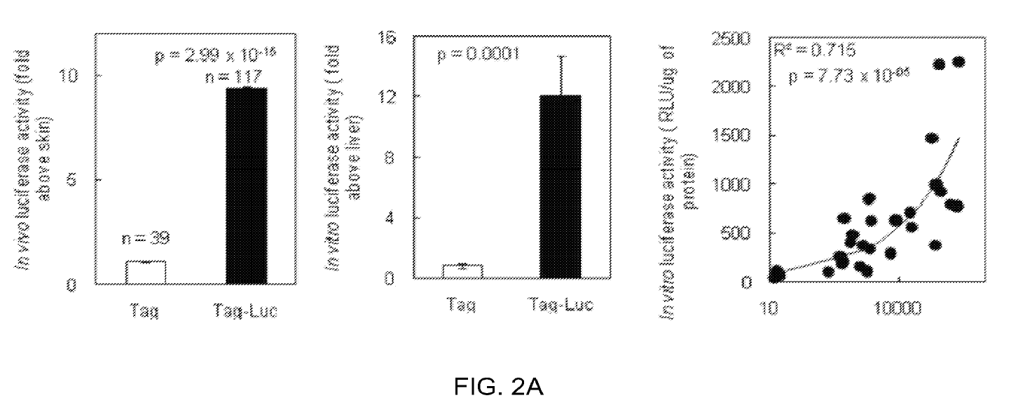

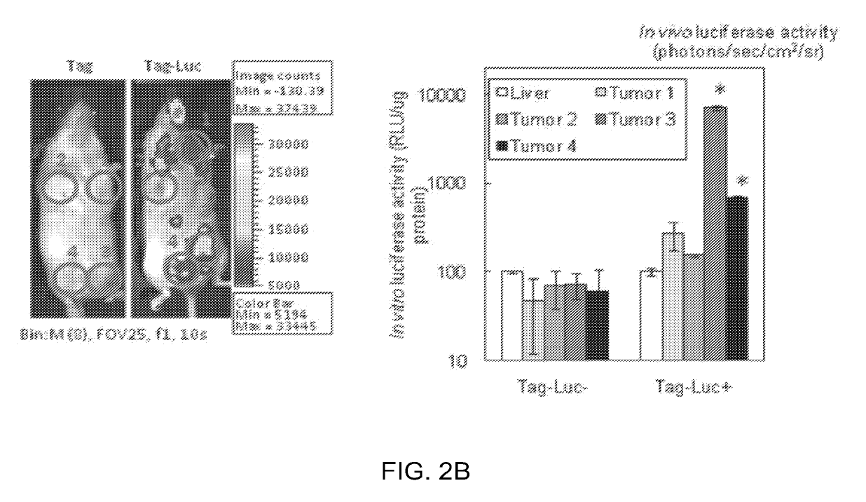

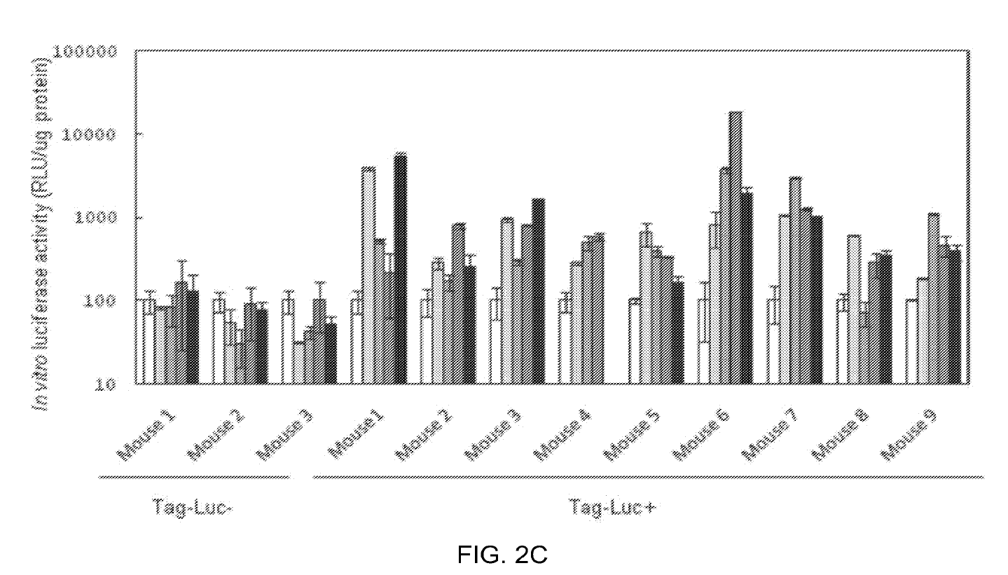

Overall, Tag-Luc tumors exhibited in vivo bioluminescent signal that was 9.36±0.08-fold greater than the background skin and significantly greater than tumors from control Tag mice (p=2.99×10⁻¹⁵; FIG. 2A, left panel). Tumors isolated ex vivo from Tag-Luc transgenic mice had 14.5-fold greater luciferase activity than tumors isolated from control Tag transgenic mice (FIG. 2A, middle panel; p=0.0001; n=16 for Tag mice, n=33 for Tag-Luc mice). To measure XMP-luc activity in these tumors, the tumors were isolated, diced into small fragments, lysed, and incubated for 20 minutes with Bright Glo lysis buffer (Promega, Madison, Wis.). Luciferase activity was detected on a Monolight 2010 (Analytic Luminescence Laboratory, San Diego, Calif.). Furthermore, in vivo bioluminescence signal of a tumor significantly correlated with in vitro luciferase activity of a tumor that had been excised and lysed (FIG. 2A, right panel; p=7.73×10⁻⁵). Overall, primary breast tumors from Tag-luc mice exhibited XBP1-luc activity that was significantly increased above background.

Different tumors within the same mouse exhibited distinct bioluminescent signals (FIG. 2B). The heterogeneity of XBP1-luc activity in different primary tumors growing in the same mouse remained a consistent phenomenon seen in many different mice studied (FIG. 2C). In contrast to Tag-Luc mice, tumors arising in Tag mice (FIG. 2B, left panel and FIG. 2C) did not possess greater bioluminescence signal than non-neoplastic tissues either in vivo or ex vivo. In FIG. 2B, left, image counts are Min −130.39, max 374.39, color bar min.=5194, max=33445. In FIG. 2B, the bars are, from left to right, Liver, Tumor 1, Tumor 2, Tumor 3 and Tumor 4.

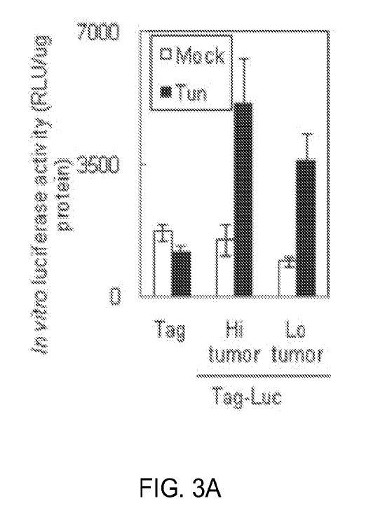

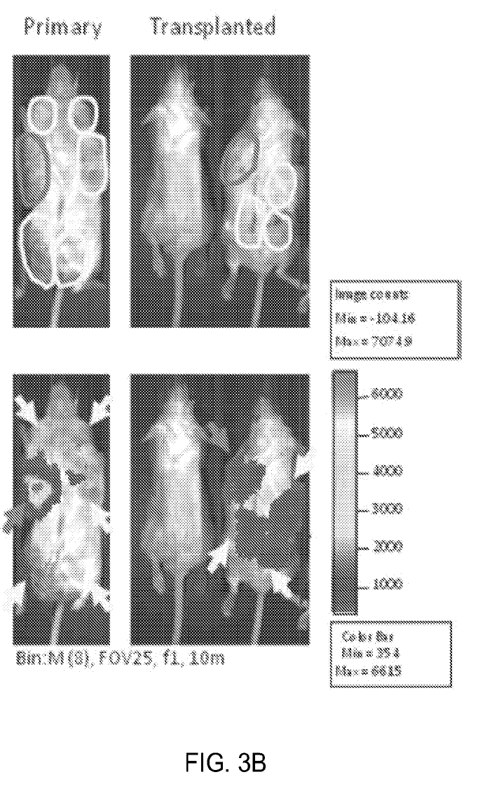

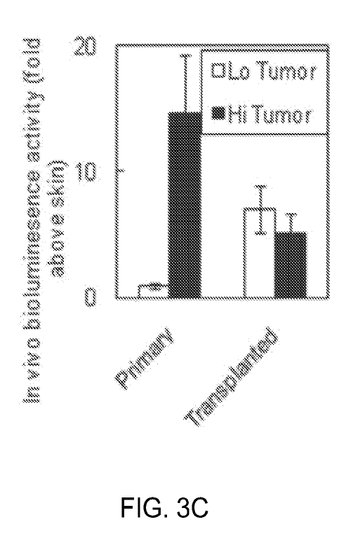

To determine whether this heterogeneous bioluminescence was a hereditary feature of the tumor, tumors with bioluminescence signal above background skin were defined as “Hi tumors” and those with bioluminescence signal similar to background skin as “Lo tumors.” In vivo differences in XBP1-luc activity was not maintained in cell culture, since both Hi and Lo tumors demonstrated background bioluminescent activity that was comparable to excised control tumors lacking the XBP1-luc reporter (FIG. 3A). Tunicamycin increased XBP1-luc activity in Hi and Lo tumors to similar levels, indicating that both tumor phenotypes retained similar responsiveness to ER stress.

To further demonstrate that primary tumors developed variable and non-hereditable levels of ER stress, primary tumors were harvested from mice bearing both Hi and Lo tumors and transplanted subcutaneously into the flanks of syngeneic mice (see FIG. 3B, Image counts, min. −104.16, max 707.49, color bar min 354, max 6615). Solid tumor fragments were generated by harvesting primary tumors from Tag-Luc transgenic mice. The tumors were washed and diced into small tumor fragments. The fragments were packed into a 1 ml tuberculin syringe and injected into a 10-gauge trochar. This allowed injection of similar fragment doses ranging from 0.25-0.3 ml. For injection of SCID mice, cancer cells were cultured from primary tumors for 7-14 days and 5×10⁵ cells were injected into the indicated flank. For primary and transplanted tumors, three orthogonal measurements (a, b & c) were made and tumor volume was calculated as follows: Tumor vol (mm³)=(a×b×c)/2.

FIG. 3B illustrates one set of transplantations. The Hi primary tumor (indicated in dark circle and arrow) was transplanted into the upper left flank while the Lo primary tumors (indicated in light circles or arrows) were transplanted in the remaining three quadrants of a single mouse. 14 days after transplantation, both Hi and Lo tumors exhibited similar levels of in vivo bioluminescent signal (see FIGS. 3B&C). Thus, the heterogeneity of in vivo tumor bioluminescence was not simply a heritable trait of each tumor and reflected the variation of microenvironmental stress in primary tumors.

### Example 5

### Increased XBP1-Luc Activity is Inversely Correlated with Glucose Utilization In Vivo

To elucidate what factors may mediate these differences in XBP1-luc activity, tumor bearing Tag-Luc mice were injected with labeled RGD peptide or 2-deoxy-d-glucose (2-DG) probes to measure angiogenesis or glucose uptake, respectively. RGD and 2-DG avidity in tumors were obtained noninvasively using spectral fluorescence imaging with the Maestro In-Vivo imaging system (CR1, Woburn, Mass.). IRDye 800CW RGD, IRDye 800CW 2-DG and the IRDye 800CW alone were obtained from Li-Cor (Lincoln, Nebr.) and injected i.v. (intravenous) into tumor bearing mice. 24 to 48 hours later, mice were imaged using near-infrared excitation/emission filter sets and images were obtained from 780 nm to 900 nm wavelengths at 10 nm intervals. Exposure time ranged from 250 to 1500 ms. Dye uptake was measured at the 800 nm wavelength.

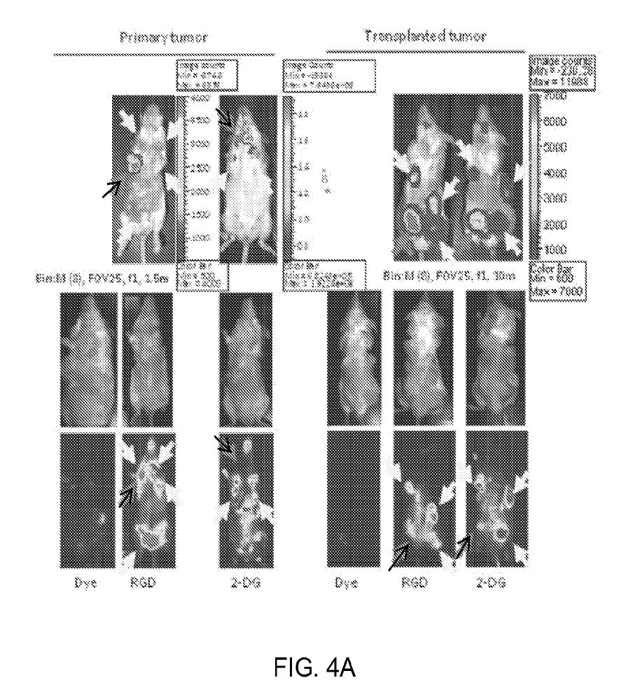

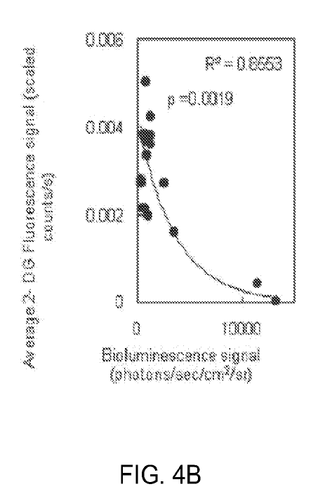

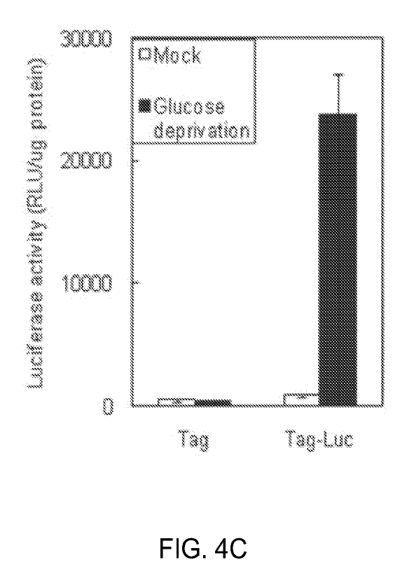

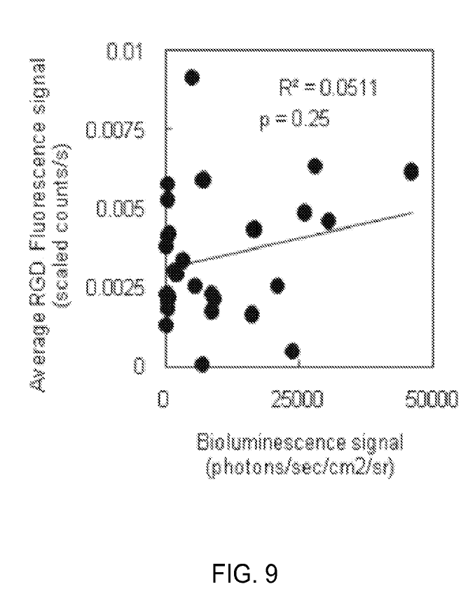

Compared to mice injected with the RGD probe or the dye alone, primary tumors with higher XBP1-luc activity (dark arrows) possessed significantly lower 2-DG uptake (FIG. 4A primary tumor, image counts min=−374.3, max=6356. left, right min=−19334, max=7,64596 e-06. color bar=min 500, max 400 (left) 1.999e-06 (right), left panels and FIG. 4B). Conversely, tumors with lower XBP1-luc activity (light arrows) had higher 2-DG uptake. By contrast, the level of XBP1-luc activity did not significantly correlate with RGD uptake in tumors (FIG. 9).

To confirm that differences in 2-DG uptake was not due to genetic differences in tumors, cells from a single Tag-Luc tumor were injected at multiple sites of a single mouse and demonstrated heterogeneous XBP1-luc signal (FIG. 4A, right panels). As with primary tumors, transplanted tumors with higher XBP1-luc activity (dark arrows) had lower 2-DG uptake. Tumors with lower XBP1-luc activity (light arrows) had higher 2-DG uptake. Thus, XBP1-luc activity in the microenvironment inversely correlated with the glucose uptake in tumors.

Since the XBP1-luc activity in tumors inversely correlated with glucose uptake, whether inhibition of glucose utilization could modulate XBP1-luc activity in primary tumors was investigated. Indeed, tumor cells cultured in glucose depleted media had 27.2-fold more luciferase activity than control treated cells (FIG. 4C). Tumors from Tag-Luc mice treated with 2-DG to mimic a state of glucose deprivation had a 1.78-fold increase in bioluminescent compared to control mice (FIG. 4D; p=0.01; n=6 for mock treated mice, n=12 for 2-DG treated mice, Image counts—min=−200.6, max=8265.9, color bar min.=500, max=2500). Therefore, the heterogeneity of ER stress in primary tumors correlated with the loss of glucose uptake.

### Example 6

### Hypoxia Modulates XBP1-Luc Signal in Spontaneous Tumors

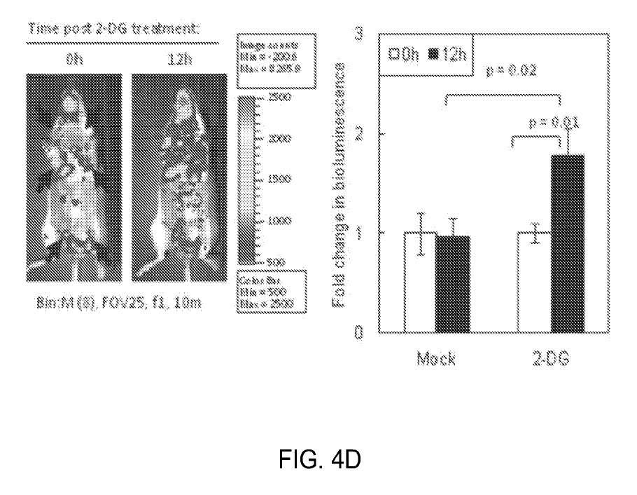

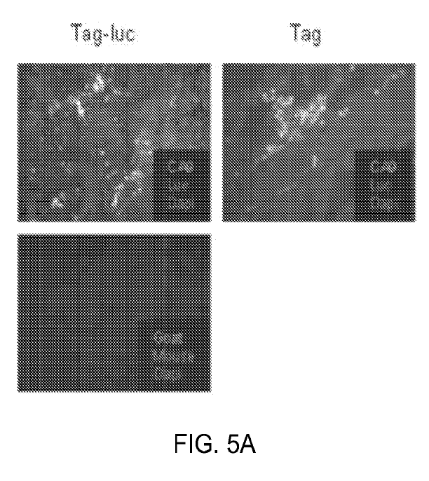

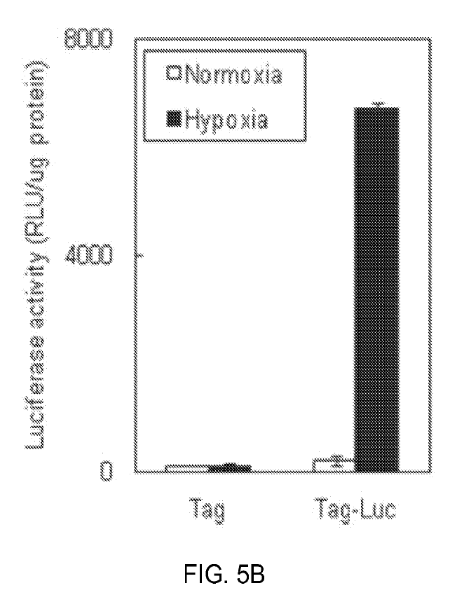

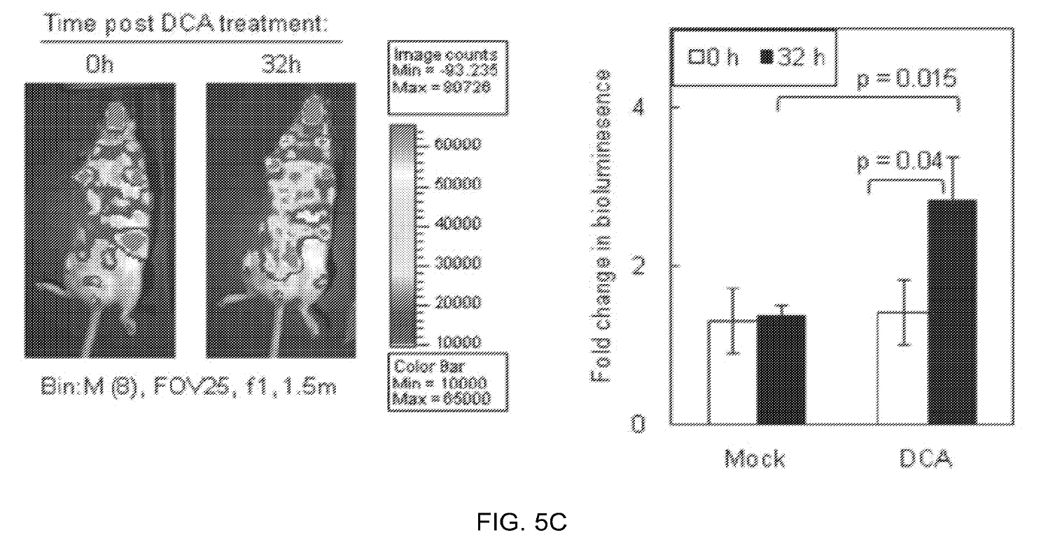

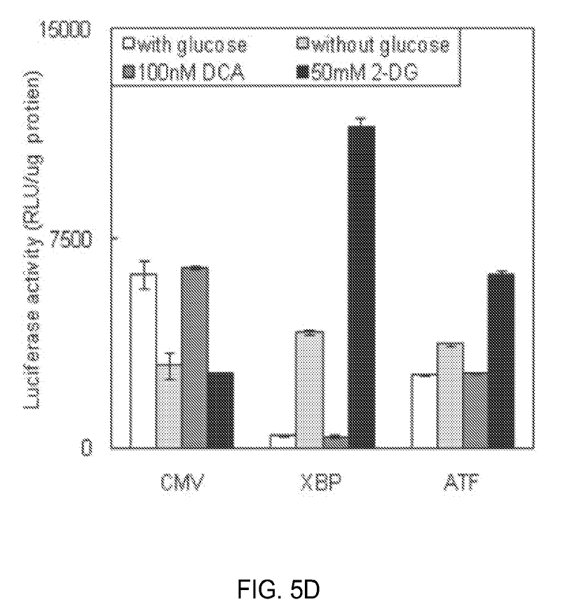

To determine if XBP1-luc activity was regulated by hypoxia, Tag-Luc tumors were analyzed for XBP1-luc protein in hypoxic areas of the tumor microenvironment. In histological tumor sections, luciferase activity colocalized with the hypoxic marker CA-9 (FIG. 5A), a HIF-1 regulated gene that is activated under moderate levels of hypoxia, indicating that XBP1 splicing occurred in hypoxic areas.

To confirm that XBP1 splicing could occur in the hypoxic microenvironments of primary tumors, primary Tag-Luc breast tumors were isolated and the cells were cultured for 5-10 days. Next, the cells were incubated for 24-48 hours under hypoxic (2% or 0.5% O₂ concentration; In vivo 400 Hypoxia Workstation, Biotrace Inc., UK) or anoxic (<0.1% O₂ concentration; Sheldon Corp., Cornelius, Oreg.) conditions. After 48 h of culturing under hypoxic conditions, Tag-Luc tumor cells exhibited 58.0-fold more luciferase activity than cells cultured under normoxic conditions (FIG. 5B, p=0.01; see also FIG. 7D and FIG. 8), indicating that Tag-Luc tumors increased levels of spliced XBP1 under conditions of increasing hypoxia.

To confirm that hypoxia regulated XBP1-luc activity, mice were treated with repeated DCA (dichloroacetic acid) injections, which increases tumor oxygen consumption, to mimic a state of increasing hypoxia. Primary Tag-Luc tumors increased bioluminescent signal by 2.0-fold after repeated DCA injections confirming that hypoxia can regulate XBP1-luc activity (FIG. 5). DCA alone did not directly induce ER stress, since HT1080 cells expressing XBP1-luc or ATF4-luc constructs did not induce UPR reporter activity in response to DCA but did induce UPR reporter activity in response to 2-DG. Therefore, hypoxic microenvironments could induce ER stress in primary tumors in vivo.

### Example 7

### Higher XBP1-Luc Activity is Correlated with Faster Doubling Times

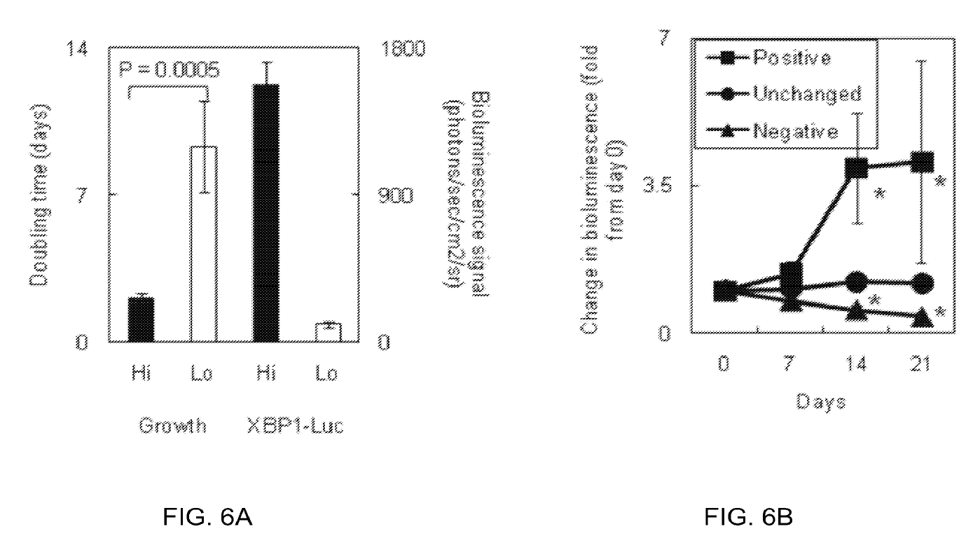

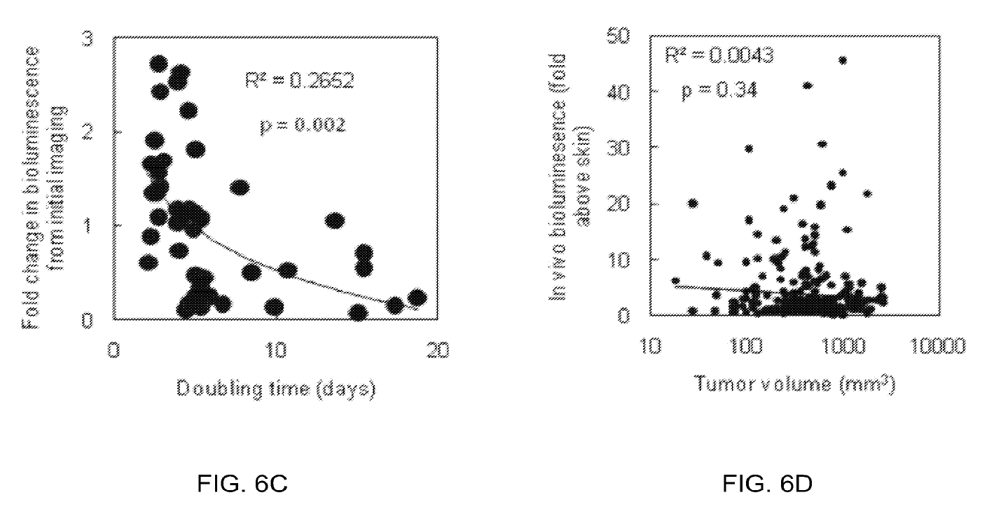

Since XBP1-luc activity was heterogeneous among similar primary tumors, the issue of whether differences in XBP1-luc activity served as a marker for differences in tumor growth rates was investigated. Tag-Luc tumors with higher XBP1-luc signal had significantly faster doubling times compared to tumors with lower XBP1-luc signal (FIG. 6A; p=0.0005). Next, Tag-Luc mice were imaged over time to detect changes in XBP1-luc signal. Three separate categories of XBP1-luc signal were observed over time: increasing signal, stable signal and decreasing signal (FIG. 6B). Compared to tumors with stable or increasing signal, tumors with decreasing signal also had slower doubling times (FIG. 6C; p=0.004). By contrast, XBP1-luc activity did not correlate with tumor size indicating that XBP1-luc activity was not simply a reflection of tumor mass (FIG. 6D).

To further characterize the relationship of tumor growth rates with XBP1-luc activity, breast cancer prone Her2 (FVB/N-Tg(MMTVneu)202Mul/J) (Guy et al. PNAS; 89: 10578-82 (1992)) mice were bred to XBP1-luc reporter mice, to yield the double transgenic Her2-Luc mouse. Compared to Tag mice, Her2 mice have a longer tumor latency period and develop slower growing tumors. Her2-Luc mice developed tumors at similar rates as the Her2 controls (FIG. 10A and Table 3). Tumors arising in Her2-Luc mice were less frequent and exhibited less intense bioluminescence signal (FIGS. 10 B&C and Table 3) than those from Tag-Luc mice. As with Tag-Luc mice, ex vivo luciferase activity of Her2-Luc tumors was statistically greater than control Her2 tumors but was significantly lower than the luciferase activity in faster growing Tag-Luc tumors. (FIG. 10D). This difference in XBP1 signal correlated with decreased growth rate of Her2 tumors as indicated by their prolonged latency and longer doubling times (Table 3). Therefore, in vivo and in vitro XBP1-luc activity was positively correlated with tumor growth rates. These data suggest that faster growing tumors likely have less time to adapt to their blood supply, leading to increased hypoxia and nutrient deprivation, microenvironment conditions consistent with increased ER stress.

TABLE 3
Characterization of mammary carcinomas in Tag and Her2 mice

| | | | | | | | Average | Total | |
|---|---|---|---|---|---|---|---|---|---|
| | | | | No. with | Tumors | Tumor | tumor | tumor | Doubling |
| | XBP- | | | tumors | per | onset | volume | volume | time |
| Mouse | Luc | Sex | No. | (frac.) | mouse | (days) | (mm³) | (mm³) | (days) |
| Tag | − | F | 13 | 13 (1) | 4.61 ± 0.38^{A} | 51.08 ± 3.67^{A} | 936.91 ± 88.12^{A} | 4252.12 ± 757.45^{A} | |
| | + | F | 23 | 23 (1) | 4.26 ± 0.23^{A} | 53.74 ± 2.39^{A} | 740.87 ± 66.90^{A} | 3124.56 ± 545.99^{A} | 4.21 ± 0.44^{E} |
| | − | M | 6 | 6 (1) | 2.83 ± 0.87^{B} | 112.33 ± 5.67^{B} | 1021.29 ± 235.88^{B} | 2893.67 ± 1236.28^{B} | |
| | + | M | 5 | 4 (0.8) | 1.6 ± 0.24^{B} | 131.33 ± 6.95^{B} | 966.12 ± 125.94^{B} | 1545.80 ± 335.34^{B} | |
| Her2 | − | F | 14 | 7 (0.5) | 1.71 ± 0.29^{C} | 235.29 ± 7.98^{C} | 1674.25 ± 383.44^{C} | 2870.14 ± 1103.74^{C} | 17.39 ± 3.35^{E} |
| | + | F | 31 | 16 (0.52) | 1.69 ± 0.22^{C} | 200.81 ± 8.62^{C} | 2266.21 ± 241.87^{C} | 3682.59 ± 612.52^{C} | |

^{A, B,} and ^{C}indicate p values >0.05 for each individual strain of mice compared using Student's a two-tailed t-test with unequal variances.
^{E}indicates p value 3.41 × 10⁻⁷

### Example 8

### XBP-1-Luc Activity is Activated in Skin During Wound Healing

Wound healing in XBP1-luc transgenic mice was studied. XBP-1-luc activity was activated robustly in the skin during wound healing, and the peak of XBP-1-luc activity was observed during the phase of wound remodeling in which collagen deposition occurs.

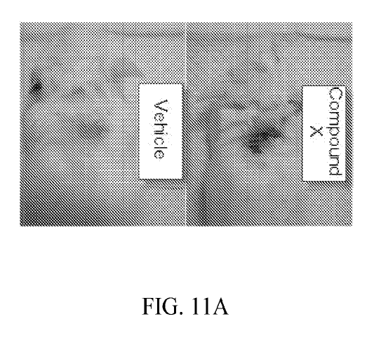

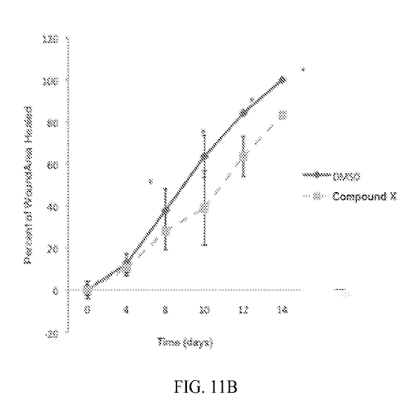

A compound capable of modulating ER stress response (Compound X) or vehicle (DMSO) was applied daily to wounds on XBP-1-luc transgenic mice. There was a significant delay in wound healing in Compound X treated mice compared to DMSO treated mice (FIG. 11A and FIG. 11B). Thus, modulation of ER stress response may be a therapeutic approach for treating wounds. Inhibitors of ER stress response may treat wounds that heal too robustly, including for example and without limitation, keloid formation or excessive scar formation. Activators of ER stress response may promote wound healing. Modulators of ER stress response can be topically or systematically administered. Transient treatment during critical phases of wound healing may be required for optimal wound healing.

### Example 9

### XBP-1-Luc Transgenic Mice for the Study of Insulin Resistance, Diabetes, and Obesity

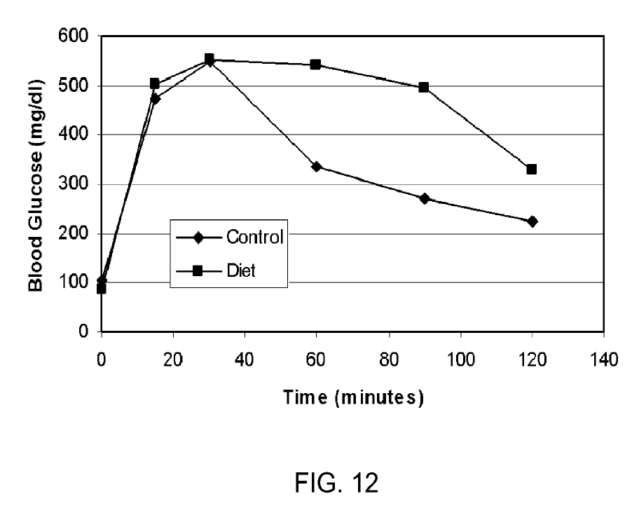

Mice were fed high fat diets to induce insulin resistance or diabetes. Insulin resistance was confirmed in mice fed a high fat diet by measuring steady state plasma glucose levels after glucose challenge in a glucose tolerance test. Mice that were fed a high fat diet (diet) compared to control mice (control), had higher levels of blood glucose in the glucose tolerance test (FIG. 12). Thus, the high fat diet induced insulin resistance and diabetes in mice.

XBP-1-luc transgenic mice fed a high fat diet can be used to study agents that modify ER stress response in vivo and to develop therapeutics for treatment of insulin resistance, diabetes, and obesity. The XBP-1-luc transgenic mice can also be bred to genetic models of diabetes, including, for example and without limitation, ob/ob mice or db/db mice, to generate diabetic XBP-1-luc transgenic mice.

Cells or organs from diabetic XBP-1-luc transgenic mice that exhibit ER stress will demonstrate increased expression of the luminogenic reporter molecule. Therapies for diabetes can be studied in mouse models of diabetes that express the XBP-1-luc transgene by detecting their effects on ER stress response.

### Example 10

### XBP-1-Luc Transgenic Mice for the Study of Cardiac Disease or Cardiomyopathy

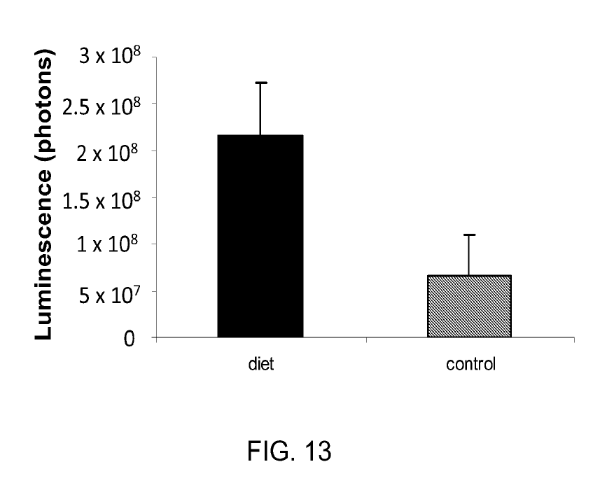

XBP-1-luc transgenic mice were fed a high fat diet. XBP-1-Luc activity was higher in the hearts of mice fed the high fat diet compared to control mice (FIG. 13). This shows that there was increased ER stress in the hearts of insulin resistant or diabetic mice.

Therapies for cardiac diseases can be studied in mouse models of cardiac disease that express the XBP-1-luc transgene by detecting their effects on ER stress response.

### Example 11

### XBP-1-Luc Transgenic Mice for the Study of Neurodegenerative Diseases

The XBP-1-luc transgene can be used to study neurodegenerative diseases, such as Alzheimer's disease, in various mouse models. The XBP-1-luc transgenic mouse can be bred to models of neurodegenerative diseases, such as Alzheimer's models, including mice that express amyloid precursor.

Cells or organs that exhibit ER stress will demonstrate increased expression of the luminogenic reporter molecule. Therapies for neurodegenerative diseases, such as Alzheimer's diseases, can be studied in mouse models of neurodegenerative disease that express the XBP-1-luc transgene by detecting their effects on ER stress response.

### Example 12

### XBP-1-Luc Transgenic Mice for the Study of Aging

The XBP-1-luc transgene can be used to study aging. XBP-1-luc transgenic mice can be aged to study ER stress response throughout their lifecycle.

Cells or organs that exhibit ER stress will demonstrate increased expression of the luminogenic reporter molecule. Anti-aging therapies can be studied in the XBP-1-luc transgenic mice by detecting their effects on ER stress response throughout the life cycles of the mice. In addition, specific organs that have ER stress response can be identified. Therapies targeting those organs can be studied by detecting the effect of the therapies on ER stress response.

### Example 13

### XBP-1-Luc Transgenic Mice for the Study of Collagen Vascular Disease

The XBP-1-luc transgene can be used to study collagen vascular disease, such as arthritis, in various mouse models. XBP-1-luc transgenic mouse in a collagen induced arthritis model can be used.

Cells or organs that exhibit ER stress will demonstrate increased expression of the luminogenic reporter molecule. Therapies for collagen vascular disease, such as arthritis, can be studied in mouse models of collagen vascular disease that express the XBP-1-luc transgene by detecting their effects on ER stress response.

### Example 14

### Modulation of the ER Response in Vivo

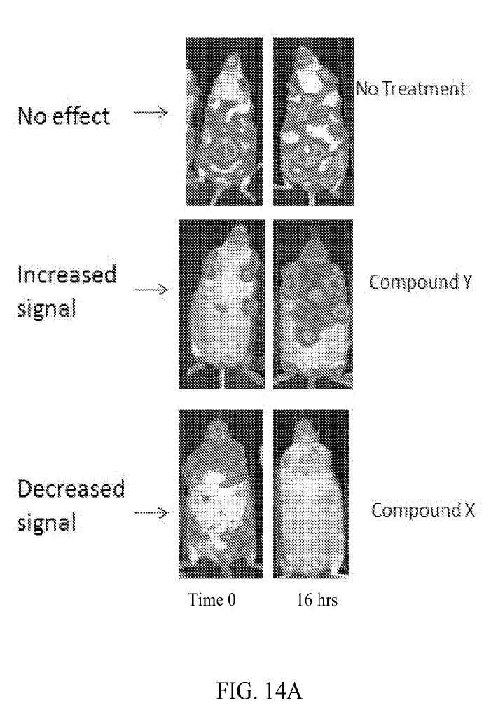

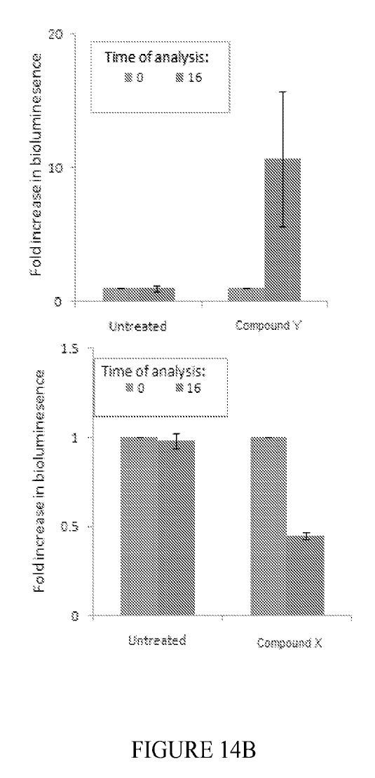

Luciferase activity was measured in vivo using the ICIS imaging system (Caliper LifeSciences Hopkinton, Mass.). Mice were injected i.p. with luciferin (300 mg/kg dissolved in PBS) and anesthetized with 3% isoflurane. Bioluminescent signal was assayed at baseline and 16 hrs after i.p. injection of either Compound Y (1 mg/kg) or Compound X (60 mg/kg). The data shows that the compounds modulated the ER response: Compound Y increased the response while Compound X decreased it. (FIG. 14A and FIG. 14B).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A transgenic animal comprising a first and second sequence, wherein the first sequence is an ER stress response regulatory sequence and the second sequence is a sequence encoding a luminogenic reporter molecule, wherein the first sequence is in operative association with the second sequence and wherein the first and second sequence are integrated in the genome of the animal's germ cell.

2. The transgenic animal of claim 1, wherein the ER stress response regulatory sequence comprises an X-box-binding protein 1 (XBP1) regulatory sequence.

3. The transgenic animal of claim 1, wherein the first and second sequence is under a heterologous promoter.

4. The transgenic animal of claim 1, wherein the first and second sequence are in a heterologous luminogenic ER stress response reporter construct.

5. The transgenic animal of claim 1, wherein the animal further comprises a mutation associated with a disease characterized by ER stress response activity.

6. The transgenic animal of claim 5, wherein the disease characterized by ER stress response activity is chosen from cancer, diabetes, cardiovascular disease, Alzheimer's disease, Parkinson's disease, amyotrophic lateral sclerosis, polyglutamine disease, prion disease, stroke, bipolar disease, atherosclerosis, wound healing, aging, arthritis, and autoimmune disease.

7. The transgenic animal of claim 6, wherein the cancer is chosen from pancreatic cancer, breast cancer, colorectal cancer, skin cancer, brain cancer, lung cancer, stomach cancer, esophageal cancer, bone cancer, colon cancer, uterine cancer, liver cancer, lymphoid cancer, ovarian cancer, rectal cancer, and thyroid cancer.

8. The transgenic animal of claim 5, wherein the mutation is K-ras (G12D).

9. The transgenic animal of claim 5, wherein the mutation is a dominant-negative mutation.

10. The transgenic animal of claim 5, wherein the mutation is a gain-of-function.

11. The transgenic animal of claim 5, wherein the mutation is a loss-of-function.

12. The transgenic animal of claim 5, wherein the mutation is a loss-of-function in a gene chosen from apoE, leptin receptor, leptin, Smad4/DPCA4, and Trp53.

13. A cell line derived from the transgenic animal of claim 1.

14. A cell line derived from the transgenic animal of claim 5.

15. A method of detecting ER stress response comprising
exposing a transgenic animal comprising a first and second sequence, wherein the first sequence is an ER stress response regulatory sequence and the second sequence is a sequence encoding a luminogenic reporter molecule, wherein the first sequence is in operative association with the second sequence and wherein the first and second sequence are integrated in the genome of the animal's germ cell to a condition suspected of modulating the ER stress response; and
detecting modulation of expression of the luminogenic reporter molecule in at least one cell to thereby detect modulation of ER stress response.

16. A method of screening a molecule for ER stress response-modulating activity comprising
administering to a transgenic animal comprising a first and second sequence, wherein the first sequence is an ER stress response regulatory sequence and the second sequence is a sequence encoding a luminogenic reporter molecule, wherein the first sequence is in operative association with the second sequence and wherein the first and second sequence are integrated in the genome of the animal's germ cell a test molecule suspected of having ER stress response-modulating activity; and
detecting expression of the luminogenic reporter molecule, wherein a change in luminogenic reporter activity in the animal treated with the test molecule, relative to an untreated control animal, is indicative of the test molecule having ER stress response-modulating activity.

17. The method of either claim 15 or claim 16, wherein the transgenic animal further comprises a mutation associated with a disease characterized by ER stress response activity.

18. A method of treating at least one disease characterized by ER stress response activity, the method comprising administering to a patient a therapeutically effective amount of at least one molecule having ER stress response-modulating activity, wherein the at least one molecule modulates ER stress response.

19. The method of claim 18, wherein the at least one molecule is identified using a method of screening a molecule for ER stress response-modulating activity comprising
administering to a transgenic animal of claim 1 a test molecule suspected of having ER stress response-modulating activity; and
detecting expression of the luminogenic reporter molecule, wherein a change in luminogenic reporter activity in the animal treated with the test molecule, relative to an untreated control animal, is indicative of the test molecule having ER stress response-modulating activity.

20. A method of detecting modulation of endoplasmic reticulum (ER) stress response, comprising
incubating a transgenic eukaryotic cell derived from a transgenic animal comprising a first and second sequence, wherein the first sequence is an ER stress response regulatory sequence and the second sequence is a sequence encoding a luminogenic reporter molecule, wherein the first sequence is in operative association with the second sequence and wherein the first and second sequence are integrated in the genome of the animal's germ cell under conditions suspected of modulating the ER stress response; and
detecting modulation of expression of the luminogenic reporter molecule to thereby detect modulation of ER stress response in the cell.

21. A method of screening a molecule for ER stress response-modulating activity, comprising
incubating a transgenic eukaryotic cell derived from a transgenic animal comprising a first and second sequence, wherein the first sequence is an ER stress response regulatory sequence and the second sequence is a sequence encoding a luminogenic reporter molecule, wherein the first sequence is in operative association with the second sequence and wherein the first and second sequence are integrated in the genome of the animal's germ cell with a test molecule suspected of having ER stress response-modulating activity; and
detecting expression of the luminogenic reporter molecule, wherein a change in luminogenic reporter activity in the cell incubated with the test molecule, relative to an untreated control cell, is indicative of the test molecule having ER stress response-modulating activity.

22. A method of detecting modulation of ER stress response, comprising
incubating a transgenic eukaryotic cell of derived from a transgenic animal comprising a first and second sequence, wherein the first sequence is an ER stress response regulatory sequence and the second sequence is a sequence encoding a luminogenic reporter molecule, wherein the first sequence is in operative association with the second sequence and wherein the first and second sequence are integrated in the genome of the animal's germ cell with a test molecule suspected of having ER stress response-modulating activity and a reference molecule; and
detecting expression of the luminogenic reporter molecule in the cell incubated with the test molecule and the reference molecule relative to a reference molecule treated control cell, to thereby detect modulation of ER stress response.

23. The method of any one of claim 20 to claim 22, wherein the cell is cultured ex vivo or in vivo.

24. The method of any one of claim 20 to claim 22, wherein the cell is cultured autogenically, allogenically, or syngenically.

25. The transgenic animal of claim 4, wherein the heterologous luminogenic ER stress response reporter construct further comprises a viral enhancer element.

26. The transgenic animal of claim 4, wherein the heterologous luminogenic ER stress response reporter construct further comprises a second heterologous promoter sequence.

27. The transgenic animal of claim 1, wherein expression of the luminogenic reporter molecule is dependent on splicing of the first sequence.

28. The transgenic animal of claim 1, wherein the ER stress response regulatory sequence comprises a sequence at least 80% identical to a fragment of at least 100 consecutive nucleotides of nucleotides 1-671 of SEQ ID NO:1, wherein the fragment comprises a sequence corresponding to nucleotides 541 to 566 of SEQ ID NO:1.

29. The transgenic animal of claim 5, wherein the mutation is the MMTV-PyVT transgene.

30. The transgenic animal of claim 5, wherein the mutation is the MMTVneu transgene.

31. The cell of claim 13, wherein the cell expresses reduced levels of one or more anti-apoptotic proteins.

32. The cell of claim 13, wherein the cell expresses increased levels of one or more pro-apoptotic proteins.

33. The transgenic animal of claim 1, wherein the luminogenic reporter molecule is coexpressed with a hypoxic marker.

34. The transgenic animal of claim 33, wherein the hypoxic marker is chosen from carbonic anhydrase 9 (CA-9), secreted phosphoprotein 1 (SPP1), thioredoxin domain containing 5 (TXNDC5), resistin like beta (RETNLB), angiopoietin-like 1 (ANGPTL1), vascular endothelial growth factor (VEGF), 78 kDa glucose upregulated protein (GRP78), 94 kDa glucose upregulated protein (GRP94), hypoxia inducible factor-1 (HIF-1), hypoxia inducible factor-2 (HIF-2), matrix metallopeptidase 9 (MMP9), osteopontin (OPN), plasminogen activator inhibitor-1 (PAI-1), and osteosarcoma amplified 9, endoplasmic reticulum lectin (OS-9).

35. The method of any one of claim 20 to claim 22, wherein the cell is cultured in the presence of a second molecule chosen from a proteasome inhibitor, brefeldin A, beta-mercaptoethanol, tunicamycin, 2-deoxy-d-glucose, dichloroacetic acid, thapsigargin, dithiothreitol, and combinations thereof.

36. A method of detecting the toxicity of a molecule comprising:
administering to a transgenic animal of claim 1 a test molecule; and
detecting expression of the luminogenic reporter molecule to thereby detect the toxicity of the molecule.

37. A method of estimating the therapeutic ratio of a molecule, the method comprising:
administering to a transgenic animal of claim 1 a test molecule;
detecting the therapeutic effect of the test molecule; and
detecting expression of the luminogenic reporter molecule, wherein the expression of the luminogenic reporter molecule is indicative of the toxicity of the test molecule, to thereby estimate the therapeutic ratio of the test molecule.
